(19) United States
(12) Patent Application Publication
(10) Pub. No.: US 2025/0075176 A1
(43) Pub. Date: Mar. 6, 2025

(54) COMPOSITIONS COMPRISING HISTONE ACETYLTRANSFERASE INHIBITORS OR REVERSE TRANSCRIPTASE INHIBITORS AND USE THEREOF

(71) Applicant: FOREVE LABS LTD., Binyamina (IL)

(72) Inventors: Michael KLUTSTEIN, Tzur Hadassa (IL); Peera WASSERZUG PASH, Tzur Hadassa (IL)

(73) Assignee: FOREVE LABS LTD., Binyamina (IL)

(21) Appl. No.: 18/830,068

(22) Filed: Sep. 10, 2024

Related U.S. Application Data
(63) Continuation of application No. PCT/IL2023/050153, filed on Feb. 14, 2023.
(60) Provisional application No. 63/317,975, filed on Mar. 9, 2022.
(60) Provisional application No. 63/436,912, filed on Jan. 4, 2023.

(51) Int. Cl. C12N 5/075

(52) U.S. Cl. CPC C12N 5/0609; C12N 2501/065; C12N 2501/31; C12N 2501/724

(57) ABSTRACT

Cell culture media comprising luteinizing hormone, follicle-stimulating hormone and at least one histone acetyltransferase inhibitor or reverse transcriptase inhibitor are provided. Methods of in vitro maturing a human oocyte, determining suitability for in vitro maturation, as well as kits comprising media, luteinizing hormone, follicle-stimulating hormone and at least one histone acetyltransferase inhibitor or reverse transcriptase inhibitor are also provided.

### CROSS REFERENCE TO RELATED APPLICATIONS

This application is a Bypass Continuation of PCT Patent Application No. PCT/IL2023/050153 having International filing date of Feb. 14, 2023, which claims the benefit of priority of U.S. Provisional Patent Application No. 63/317,975 filed on Mar. 9, 2022, and U.S. Provisional Patent Application No. 63/436,912 filed on Jan. 4, 2023, all of which are hereby incorporated by reference in their entirety.

### FIELD OF INVENTION

The present invention is in the field of in vitro oocyte maturation and fertilization.

### BACKGROUND OF THE INVENTION

Reproductive aging is characterized as age-related loss of fertility as a result of increased damage to the reproductive system and other systems. Oocytes accumulate damage in an age-related manner and deteriorate to the point where they are unable to be fertilized, do not mature, or do not generate the proper environment for early embryogenesis. In female humans this occurs at a relatively early age before the onset of aging in other organs and tissues. From the 1970s, the average childbearing age in the US (and other countries) has increased from 21 to more than 28, with many more women having children in their 40s (CDC/NCHS data). This worldwide rise has occurred over an extremely short period of time, bringing with it an overall decrease in woman's fertility rates (3.5 children per woman in 1960 to 1.8 today in the US), and a rise in the occurrence of aneuploidy related syndromes, infertility and infertility-related syndromes (such as primary ovarian insufficiency, POI). For example, a rise in miscarriages has been documented in Sweden between 2003 and 2012 from 40 per 100000 cases to 73 cases, and a rise of almost twice the number of IVF cases has been documented between 1997 and 2009 in the US. Therefore, it is crucial to understand the mechanisms underlying reproductive aging to enhance the survival of the aged oocytes and give hope of parenthood to millions of patients worldwide.

Oocyte aging is a multifactorial phenomenon. Some of the factors that were found to be associated with oocyte aging are reactive oxygen species (ROS) related damage, accumulated DNA damage, changes in the hormonal and regulatory environment of the oocyte with age, and the loss of cohesion with age causing oocyte aneuploidy.

Changes in epigenetic regulation of gene expression and altered chromosome dynamics have been recognized as contributors to aging, and epigenetic changes during aging have been listed among the “hallmarks of aging”. The loss of heterochromatin histone marks has been associated with the aging process in many systems and tissues. The consequences of heterochromatin deregulation in aging are related to the de-repression of many genes, but also to the activated transcription of transposable elements (TEs), which constitute circa 40% of the genome. The elevated expression of TEs causes significant DNA damage which has a negative effect on genome stability and cellular integrity. This has been shown to occur in several aging organisms and systems. However, little is known about the epigenetic changes that oocytes undergo with age. A new method for improving oocyte maturation and in vitro conception is therefore greatly needed.

### SUMMARY OF THE INVENTION

The present invention provides cell culture media comprising luteinizing hormone, follicle-stimulating hormone and at least one histone acetyltransferase (HAT) inhibitor or reverse transcriptase (RT) inhibitor. Methods of in vitro maturing a human oocyte and determining suitability for in vitro maturation (IVM) are also provided. Kits comprising media, luteinizing hormone, follicle-stimulating hormone and at least one histone acetyltransferase inhibitor or reverse transcriptase inhibitor are also provided.

According to a first aspect, there is provided a cell culture medium comprising luteinizing hormone (LH), follicle-stimulating hormone (FSH) and at least one histone acetyltransferase (HAT) inhibitor or reverse transcriptase (RT) inhibitor.

According to some embodiments, the medium is human oocyte in vitro maturation (IVM) medium comprising at least HAT or RT inhibitor.

According to some embodiments, the HAT is selected from E1A-associated protein p300 (p300), Cyclic adenosine monophosphate Response Element Binding protein Binding Protein (CREBBP/CBP), Histone acetyltransferase KAT5 (KAT5/Tip60), KAT6A, and N-acetyltransferase 10 (NAT10).

According to some embodiments, the HAT inhibitor is not a P300/CBP-associated factor (PCAF/KAT2B) inhibitor.

According to some embodiments, the HAT inhibitor is selected from the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, Epigallocatechin gallate (EGCG), anacardic acid, Gambogic acid, Delphinidin, Remodelin, butyrolactone 3 (MB-3), NK13650A, Taxodione (NP-15), Δ12-prostaglandin J2 (PGJ2), Plumbagin (RTK1), NU9056, DC-M01-7, CCT077791, NSC694614, PU141, C646, TH1834, L002, WM-8014, DC-G16-11, and derivatives thereof.

According to some embodiments, the HAT inhibitor is a naturally occurring molecule.

According to some embodiments, the HAT inhibitor is selected from curcumin, plumbagin and garcinol.

According to some embodiments, the RT inhibitor is selected from the group consisting of zidovudine, didanosine, zalcitabine, stavudine, lamivudine, abacavir, emtricitabine, entecavir, truvada, azvudine, tenofovir, adefovir, efavirenz, nevirapine, delavirdine, etravirine, rilpivirine, doravirine, islatravir and derivatives thereof.

According to some embodiments, the RT inhibitor is selected from zidovudine (azidothymidine, AZT) and lamivudine (3TC).

According to some embodiments, the medium comprises both a HAT inhibitor and an RT inhibitor.

According to some embodiments, the medium comprises curcumin and AZT.

According to some embodiments, the curcumin is present at a concentration of about 5-20 μM.

According to some embodiments, the curcumin is present at a concentration of about 10 μM.

According to some embodiments, the AZT is present at a concentration of about 0.5-5 μM.

According to some embodiments, the AZT is present at a concentration of about 1 μM.

According to some embodiments, the cell culture medium comprises a pH of between 6.8 and 7.8.

According to some embodiments, the medium is for use in IVM of a human oocyte.

According to some embodiments, the human oocyte was retrieved from a 35 year or older female.

According to another aspect, there is provided a method of in vitro maturing a human oocyte, the method comprising receiving an immature oocyte from a human subject and culturing the received immature oocyte in culture medium comprising at least one HAT inhibitor or RT inhibitor for a time sufficient for first polar body extrusion from the oocyte, thereby in vitro maturing a human oocyte.

According to some embodiments, the human subject is at least 35 years or older.

According to some embodiments, the human subject is at least 38 years or older.

According to some embodiments, the human subject does not receive hormone injections before harvesting of the immature oocyte.

According to some embodiments, the hormone injections comprise injection of at least one of FSH, LH, human menopausal gonadotrophin (HMG) and human chorionic gonadotropin (HCG).

According to some embodiments, the culture media is IVM culture media supplemented with at least one gonadotropin.

According to some embodiments, the IVM culture media is supplemented with LH and FSH.

According to some embodiments, the HAT is selected from E1A-associated protein p300 (p300), Cyclic adenosine monophosphate Response Element Binding protein Binding Protein (CREBBP/CBP), Histone acetyltransferase KAT5 (KAT5/Tip60), KAT6A, and N-acetyltransferase 10 (NAT10).

According to some embodiments, the HAT inhibitor is not a P300/CBP-associated factor (PCAF/KAT2B) inhibitor.

According to some embodiments, the HAT inhibitor is selected from the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, Epigallocatechin gallate (EGCG), anacardic acid, Gambogic acid, Delphinidin, Remodelin, butyrolactone 3 (MB-3), NK13650A, Taxodione (NP-15), Δ12-prostaglandin J2 (PGJ2), Plumbagin (RTK1), NU9056, DC-M01-7, CCT077791, NSC694614, PU141, C646, TH1834, L002, WM-8014, DC-G16-11, and derivatives thereof.

According to some embodiments, the HAT inhibitor is a naturally occurring molecule.

According to some embodiments, the HAT inhibitor is selected from curcumin, plumbagin and garcinol. According to some embodiments, the HAT inhibitor is selected from curcumin and plumbagin.

According to some embodiments, the RT inhibitor is selected from zidovudine, didanosine, zalcitabine, stavudine, lamivudine, abacavir, emtricitabine, entecavir, truvada, azvudine, tenofovir, adefovir, efavirenz, nevirapine, delavirdine, etravirine, rilpivirine, doravirine, and islatravir.

According to some embodiments, the RT inhibitor is selected from zidovudine (azidothymidine, AZT) and lamivudine (3TC).

According to some embodiments, the culture media comprises both a HAT inhibitor and an RT inhibitor.

According to some embodiments, the culture media comprises curcumin and AZT.

According to some embodiments, the culture media comprises a pH of between 6.8 and 7.8.

According to some embodiments, the culture medium is the culture medium of the invention.

According to some embodiments, the time is about 18-72 hours.

According to some embodiments, the HAT inhibitor is present at a concentration sufficient to increase constitutive heterochromatin levels in the oocyte.

According to some embodiments, the constitutive heterochromatin levels are increased by at least a predetermined threshold.

According to some embodiments, the predetermined threshold is an increase of 25%.

According to some embodiments, the HAT inhibitor or RT inhibitor is present at a concentration sufficient to decrease retrotransposon expression in the oocyte.

According to some embodiments, the retrotransposon expression is decreased by at least a predetermined threshold.

According to some embodiments, the predetermined threshold is a decrease of 25%.

According to some embodiments, the culturing further comprises the addition of a meiosis entry inhibitor.

According to some embodiments, the method elevates maturation rates as compared to IVM performed in the absence of the HAT inhibitor or the RT inhibitor.

According to some embodiments, the HAT inhibitor or RT inhibitor is present at a concentration sufficient to increase first polar body extrusion as compared to culturing in media devoid of the HAT inhibitor or RT inhibitor.

According to another aspect, there is provided a method of determining suitability of a subject in need of IVM to be treated by a method of the invention, the method comprising:
a. receiving from a human female subject at least one germinal vesicle (GV)-arrested oocyte; and
b. measuring heterochromatin in the GV-arrested oocyte, wherein a heterochromatin level below a predetermined threshold indicates the subject is suitable for IVM by a method of the invention;
   thereby determining the suitability of a subject to be treated by a method of the invention.

According to some embodiments, the GV-arrested oocyte was harvested for in vitro fertilization (IVF) but was found insufficiently mature.

According to some embodiments, the measuring heterochromatin comprises measuring histone 3 lysine 9 (H3K9) trimethylation, H4K20 trimethylation, heterochromatin protein 1 (HP1) levels, 5 methylcytosine levels or a combination thereof.

According to some embodiments, the subject has undergone at least one unsuccessful round of IVF and heterochromatin levels above the predetermined threshold indicate the subject is suitable for another round of IVF.

According to another aspect, there is provided a kit comprising:
a. cell culture medium;
b. luteinizing hormone (LH) and follicle-stimulating hormone (FSH); and
c. at least one histone acetyltransferase (HAT) inhibitor or reverse transcriptase (RT) inhibitor.

According to some embodiments, the medium is human oocyte in vitro maturation (IVM) medium.

According to some embodiments, the HAT is selected from E1A-associated protein p300 (p300), Cyclic adenosine monophosphate Response Element Binding protein Binding Protein (CREBBP/CBP), Histone acetyltransferase KAT5 (KAT5/Tip60), KAT6A, and N-acetyltransferase 10 (NAT10).

According to some embodiments, the HAT inhibitor is not a P300/CBP-associated factor (PCAF/KAT2B) inhibitor.

According to some embodiments, the HAT inhibitor is selected from the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, Epigallocatechin gallate (EGCG), anacardic acid, Gambogic acid, Delphinidin, Remodelin, butyrolactone 3 (MB-3), NK13650A, Taxodione (NP-15), Δ12-prostaglandin J2 (PGJ2), Plumbagin (RTK1), NU9056, DC-M01-7, CCT077791, NSC694614, PU141, C646, TH1834, L002, WM-8014, DC-G16-11, and derivatives thereof.

According to some embodiments, the HAT inhibitor is a naturally occurring molecule.

According to some embodiments, the HAT inhibitor is selected from curcumin, plumbagin and garcinol.

According to some embodiments, the RT inhibitor is selected from the group consisting of: zidovudine, didanosine, zalcitabine, stavudine, lamivudine, abacavir, emtricitabine, entecavir, truvada, azvudine, tenofovir, adefovir, efavirenz, nevirapine, delavirdine, etravirine, rilpivirine, doravirine, islatravir and derivatives thereof.

According to some embodiments, the RT inhibitor is selected from zidovudine (azidothymidine, AZT) and lamivudine (3TC).

According to some embodiments, the kit comprises both a HAT inhibitor and an RT inhibitor.

According to some embodiments, the kit comprises curcumin and AZT.

According to some embodiments, the cell culture medium comprises a pH of between 6.8 and 7.8 and is sufficiently buffered such that addition of the HAT inhibitor or the RT inhibitor does not reduce the pH to below 6.8.

According to some embodiments, the kit is for use in IVM of a human oocyte.

According to some embodiments, the human oocyte was retrieved from a 35 year or older female.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C: (1A) 9-month-old oocytes show maturation defects but are not uneuploid: In vitro maturation outcomes of naïve young (2-month-old) and old (9-month-old) oocytes, collected at Prophase I arrest and released simultaneously to meiosis. Oocytes were fixed and stained for DNA to assess entry into MII (Z test for 2 proportions p=0.00135, animals used for this experiment N_{young}=3, N_{old}=3), see Methods. (1B) Young (2-month-old) and old (9-month-old) oocytes chromosomes were spread at prophase of MII. Sister chromatid pairs were counted for each cell to assess ploidy. (Z test p=0.554, animal used for experiment N_{young}=3, N_{old}=4) (1C) Representative image of metaphase II chromosome spread, with and without CREST staining.

FIGS. 2A-2D: 9-month-old oocytes lose heterochromatin marks: (2A-2D) In situ staining images and staining intensity analysis for chromatin marks and proteins in oocytes from young (2-month-old) and old (9-month-old) females. Oocytes were stained and imaged during prophase I arrest by in situ immunofluorescence (see Methods). Intensity of staining was quantified for each image, and statistical analysis was performed on the numerical values (see Methods). Constitutive heterochromatin marks and proteins were measured by staining for (2A) H3K9me2 (t test p<0.0001, animals used for experiment N_{young}=4, N_{old}=4) and (2B) HP1γ(t test p=0.0146, animals used for experiment N_{young}=3, N_{old}=3). (2C) To examine facultative heterochromatin oocyte were stained and imaged for H3K27me3 (t test p=0.0005, animals used for experiment N_{young}=3, N_{old}=5) (2D) Euchromatin staining was performed by staining for the marker H3K4me3 (t test p32 0.657, animals used for experiment N_{young}=3, N_{old}=6).

FIGS. 3A-3D: Heterochromatin loss appears uniformly in diverse oocyte populations: IHF staining was performed on ovaries cryosections from young (2-month-old) and old (9-month-old) female mice. Loss of heterochromatin was detected in the intact oocytes in both (3A) H3K9me2 (t test<0.0001, animals used for experiment N_{young}=3, N_{old}=3) and (3B) H3K27me3 staining. (MW=0.0008, animals used for experiment N_{young}=3, N_{old}=3). Staining of MI metaphase chromosomes and staining intensity analysis from young (2-month-old) and old (9-month-old) females (see Methods) was performed for the following markers: (3C) H3K9me2 (t test p=0.008, animals used for experiment N_{young}=3, N_{old}=5) and (3D) H3K27me3 (MW p<0.0001, animals used for experiment N_{young}=3, N_{old}=4).

FIGS. 4A-4H: Old oocytes show elevated activity of retrotransposons L1 and IAP and increased DNA damage: (4A) Transcriptional retrotransposon activity was measured using qRT-PCR for L1 retrotransposon (MW p=0.01, animals used for experiment N_{young}=10, N_{old}=10) and IAP elements mRNA (MW p=0.03, animals used for experiment N_{young}=10, N_{old}=10) (see Methods) in young (2-month-old) and old (9-month-old) prophase I-arrested mouse oocytes. (4B) Upregulation of L1 retrotransposon protein was measured by Western blotting for L1-ORF1p in from young (2-month-old) and old (9-month-old) prophase I-arrested mouse oocytes. (N_{young}=6, N_{old}=8) (4C) Upregulation of L1 retrotransposon activity was measured by in situ staining for L1-ORF1p in from young (2-month-old) and old (9-month-old) prophase I-arrested mouse oocytes. (Shapiro Wilk p<0.05, t test p<0.0001, N_{young}=3, N_{old}=3). (4D) Accumulation of DNA damage with age was assessed by in situ staining for Rad51 and quantification of its nuclear localization (see Methods) in young (2-month-old) and old (9-month-old) prophase I-arrested mouse oocytes (MW p<0.0005, N_{young}=3, N_{old}=5). (4E) Another measure for DNA damage was performed by in situ staining for γH2AX (MW p=0.0085, N_{young}=3, N_{old}=5) that was quantified by the number of nuclear foci in old and young prophase I-arrested mouse oocytes (see Methods). (4F) To investigate the phenotype of aged oocytes after retroviral activity inhibition, oocytes were treated with AZT and then stained for γH2A and the number of nuclear foci in AZT treated and untreated control old prophase I-arrested oocytes was quantified (MW p<0.0274 animals used for experiment N=3) (4G) The maturation efficiency of old oocytes treated with two different doses of AZT and matured in vitro was measured by proportion of oocytes which successfully completed MII after treatment (Z test p=0.0102) (see Methods). Because incubation with the drug required longer incubation in IBMX than normal, an extended incubation without AZT was also added as control (4H) Quantification of in situ staining intensity for H3K9me2 in control old oocytes and old oocytes treated with two different doses of AZT (see Methods).

FIGS. 5A-5E: Old oocytes show elevated retrotransposon RNA processing activity: Small RNA-seq was performed in order to quantify the differential presence of small RNAs in young (2-month-old) and old (9-month-old) oocytes (see Methods). (5A) Identification of 10 repeat types that are most upregulated or downregulated in old oocytes. Note that the predominant types of repeats that were overexpressed are retrotransposon RNA molecules, while the list of under-expressed repeat types also includes other types of repeats. (5B) Family based analysis of smRNA. The ratio in sequencing reads between old and young in the transcripts of each repeat family are plotted. tRNA and rRNA are used as controls. (5C-5D) To quantify global RNA processing activity in the aged oocytes, we stained for (5C) dsRNA (MW p<0.0001, N_{young}=3, N_{old}=5) and (5D) Dicer (MW p<0.0001, N_{young}=3, N_{old}=5) in young and old prophase I-arrested mouse oocytes. (5E) Dicer elevation was confirmed using Western blotting (N_{young}=6, N_{old}=8).

FIGS. 6A-6H: Human oocytes show a reduction of the H3K9me2 heterochromatin marker, and REC8 meiotic cohesion marker with age: To study the changes in heterochromatin with age in Human oocytes, human GV-arrested oocytes were stained by immunofluorescence for H3K9me2 (see text and Methods). Oocytes were retrieved from 33 women, with an average of 2.2 oocytes per woman. Analysis was performed separately for oocytes that were stained during prophase I arrest or arrested later during meiosis. (6A) Examples of stained (for H3K9me2) prophase I-arrested oocytes from patients under 35-year-old and over 35-year-old. (6B) Oocytes arrested during meiosis also show a reduction of signal intensity with age with a linear correlation, (N=28, F test p=0.0008). The oocytes of each patient are in a different color (for a given age, several patients with more than one oocyte may be represented). (6C) Examples of stained (for H3K9me2) prophase I oocytes after meiotic resumption, from patients under 35-year-old and over 35-year-old. (6D) Prophase I oocytes show a reduction of signal intensity with age with a linear correlation. (N=21, F test p=0.03) (6E) Examples of in situ staining of prophase I-arrested human oocytes (N=21) for Rec8 meiotic cohesion subunit. (6F) These oocytes show a linear reduction of signal intensity with age (F test p=0.02). (6G) Examples of in situ staining of human prophase I oocytes after meiotic resumption (N=47) for Rec8. (6H) These oocytes show a linear reduction of signal intensity with age (F test p=0.01).

FIGS. 7A-7C: Treatment of oocytes with Chaetocin mimics natural aging: Young prophase I-arrested mouse oocytes (9 animals were used for this experiment) were released to resume meiosis in a medium supplemented with Chaetocin that inhibits heterochromatin formation (see Methods). (7A) The resulting phenotype was measured by the assessment of maturation efficiency of treated oocytes compared with untreated controls (Z test p=0.008). Incubation with Chaetocin requires sealing of the plate for bio-safety reasons. Therefore, an additional control with a sealed plate but without the drug was added (see Methods). (7B) Activation of L1 retrotransposons following Chaetocin treatment was measured by immunofluorescence for L1-ORF1p in young MII mouse oocytes with or without Chaetocin (MW p=0.0007). (7C) To measure RNA processing activity in Chaetocin-treated oocytes, in situ staining was performed for dsRNA in young MII mouse oocytes with or without Chaetocin. (MW p=0.02).

FIGS. 8A-8H: Aged oocytes recovery occurs when heterochromatin is enhanced: Overexpression of chromatin modifier enzymes was performed in old (9-month-old) prophase I-arrested oocytes. (8A) Heterochromatin recovery in EZH2 electroporated oocytes was measured by H3K27me3 staining (MW p=0.03, 4 animals used for experiment) (8B) Reduction in retrotransposon activity in treated oocytes was observed by staining for L1-ORF1 (MW p=0.03, 3 animals used for experiment). (8C) Cellular functionality was measured by successful MII accomplishment in EZH2 electroporated oocytes compared with empty electroporation control. (Z test p=0.019, 7 animals used for experiment) (8D) Heterochromatin recovery in SIRT1 electroporated oocytes was measured by H3K9me2 staining (MW p=0.0026 3 animals used for experiment). (8E) Reduction in retrotransposon activity in treated oocytes was observed by staining for L1-ORF1 (MW p=0.0013 5 animals used for experiment). (8F) Cellular functionality was measured by successful MII accomplishment in SIRT1 electroporated oocytes compared with empty electroporation control. (Z test p=0.0086, 7 animals used for experiment) (8G) Quantification of staining intensity for H3K9me2 in control old oocytes and old oocytes treated with SRT-1720 (MW p=0.0079) (8H) Maturation efficiency of old oocytes treated with SRT-1720 and matured in vitro was measured by the proportion of oocytes which succeed to complete MI after treatment. (Z test p=0.0078, animals used for experiment N=6). As above, since incubation with the drug required longer IVM incubation than normal, an extended incubation without SRT-1720 was also added as control.

FIGS. 9A-9D: Bar graphs of maturation rates in IVM culture of old mouse oocytes supplemented with (9A) Curcumin, (9B) Garcinol, (9C) various HAT inhibitors and one RT inhibitor and (9D) HAT inhibitors or a combination of curcumin and AZT.

FIGS. 10A-10B: (10A-10B) In situ staining in old and young (10A) GV and (10B) GVBD human oocytes for H3K9me2 (Left). Line graphs of biological age as compared to H3K9me2 staining intensity and regression lines (GV pval=0.03, GBVD pval=0.0008) showing an inverse correlation are provided (Right).

FIG. 11: Bar graph of oocyte maturation of oocytes from a 20-year-old fertility preservation patient in IVM protocol with AZT supplementation.

### DETAILED DESCRIPTION OF THE INVENTION

The present invention, in some embodiments, provides cell culture media comprising luteinizing hormone, follicle-stimulating hormone and at least one histone acetyltransferase inhibitor or reverse transcriptase inhibitor. Methods of in vitro maturing a human oocyte and determining suitability for in vitro maturation are also provided.

### Methods of In Vitro Maturation

By a first aspect, there is provided a method of maturing an oocyte, the method comprising receiving an immature oocyte and culturing the received immature oocyte in medium comprising at least one histone acetyltransferase (HAT) HAT inhibitor, reverse transcriptase (RT) inhibitor or both, thereby maturing an oocyte.

In some embodiments, the method is an in vitro method. In some embodiments, the method is an ex vivo method. In some embodiments, the method is performed in culture. In some embodiments, the method is a method of in vitro maturation (IVM). In some embodiments, the method is part of in vitro fertilization (IVF). In some embodiments, the method further produces a mature oocyte. In some embodiments, the mature oocyte is suitable for fertilization. In some embodiments, the mature oocyte is suitable for IVF. In some embodiments, the method further comprises fertilizing the mature oocyte. In some embodiments, the method further comprises transferring the fertilized mature oocyte to a female. In some embodiments, the method further comprises IVF. In some embodiments, the mature oocyte is for use in IVF. In some embodiments, the fertilized mature oocyte is for use in transplantation to a female.

The term oocyte refers to the female gamete before fertilization. In some embodiments, the oocyte is a mammalian oocyte. In some embodiments, the mammal is a human. In some embodiments, the subject is an older subject. In some embodiments, the subject is reproductively an older subject. In some embodiments, the human subject is at least 30, 31, 32, 33, 34, 35, 36, 37, 38, 39 or 40 years old. Each possibility represents a separate embodiment of the invention. In some embodiments, the human subject is at least 35 years old. In some embodiments, the human subject is at least 38 years old. In some embodiments, the human subject is at least 40 years old.

In some embodiments, the oocyte is at least one oocyte. In some embodiments, the oocyte is a plurality of oocytes. In some embodiments, the oocyte is a cumulus oocyte complex. In some embodiments, the oocyte is at least 1, 2, 3, 4, 5, 6, 7, 8, 9 or 10 oocytes. Each possibility represents a separate embodiment of the invention. In some embodiments, the oocyte is 1-40, 5-40, 10-40, 1-30, 5-30, 5-10, 10-40 or 10-30 oocytes. Each possibility represents a separate embodiment of the invention. In some embodiments, the oocyte is 1-40 oocytes.

In some embodiments, the method comprises extracting the immature oocyte from the subject. In some embodiments, extracting is harvesting. In some embodiments, the subject receives hormones before the extracting. In some embodiments, the subject does not receive hormone injections before the harvesting. In some embodiments, the immature oocyte is from a subject that has received hormone injections. In some embodiments, the immature oocyte is from a subject that has not received hormone injections. In some embodiments, the hormone is a hormone that induces maturation. In some embodiments, the hormone is a gonadotropin. In some embodiments, the hormone is selected from follicle stimulating hormone (FSH), luteinizing hormone (LH), human menopausal gonadotrophin (HMG), human chorionic gonadotropin (HCG) and a combination thereof. In some embodiments, the hormone comprises or is FSH. In some embodiments, the hormone comprises or is LH. In some embodiments, the hormone comprises or is HMG In some embodiments, the hormone comprises or is HCG.

In some embodiments, an immature oocyte is an oocyte that has not reached meiotic competency. In some embodiments, an immature oocyte is an oocyte that has not reached the first stages of meiotic division. In some embodiments, an immature oocyte is an oocyte that has not divided into MI. In some embodiments, an immature oocyte is an oocyte without a polar body. In some embodiments, an immature oocyte is an MI oocyte. In some embodiments, an immature oocyte is a germinal vesicle oocyte. In some embodiments, an immature oocyte is a cumulus enclosed oocyte. In some embodiments, an immature oocyte is a cumulus-oocyte complex. In some embodiments, an immature oocyte is an oocyte not suitable for fertilization.

In some embodiments, the culturing is in medium. In some embodiments, the medium is tissue culture medium. In some embodiments, the culturing is tissue culturing. In some embodiments, the culturing is in vitro culturing. In some embodiments, the medium is IVM medium. In some embodiments, the medium is IVF medium. IVM and IVF media are well known in the art and commercially available. Any such media may be used as part of the invention. Examples of IVM media include, but are not limited to, SAGE in-vitro maturation (IVM) media, MediCult, human tubal fluid, and blastocyst culture medium. In some embodiments, the medium is SAGE medium. In some embodiments, the SAGE medium is SAGE IVM medium.

In some embodiments, the medium is supplemented with at least one hormone. In some embodiments, the hormone is a gonadotropin. In some embodiments, the gonadotropin is LH. In some embodiments, the gonadotropin is FSH. In some embodiments, the medium is supplemented with LH and FSH. In some embodiments, the LH is supplemented at a concentration of 0.075 IU/mL. In some embodiments, the FSH is supplemented at a concentration of 0.075 IU/mL.

Histone acetyltransferases are well known in the art and lists of known or putative HATs can be found for example in Sterner and Berger, “Acetylation of Histones and Transcription-Related Factors”, Microbiol Mol Biol Rev. 2000 June; 64(2): 435-459, herein incorporated by reference in its entirety. There are two major HAT families: the Gen5-related N-acetyltransferases (GNATs) family, and the MYST HAT family. Some HATs however belong to neither family, such as p300, CBP, nuclear receptor coactivators (e.g., ACTR/SRC-1), TAFII250, TFIIIC, Rtt 109, and CLOCK. In some embodiments, the HAT is E1A-associated protein p300 (p300). In some embodiments, the HAT is Cyclic adenosine monophosphate Response Element Binding protein Binding Protein (CREBBP/CBP). In some embodiments, the HAT is Histone acetyltransferase KAT5 (KAT5/Tip60). In some embodiments, the HAT is KAT6A. In some embodiments, the HAT is N-acetyltransferase 10 (NAT10). In some embodiments, the HAT is steroid-receptor coactivator (SRC-1). In some embodiments, the HAT is ACTR. In some embodiments, the HAT is TAFII250. In some embodiments, the HAT is TFIIIC. In some embodiments, the HAT is Rtt 109. In some embodiments, the HAT is CLOCK. In some embodiments, the HAT is not P300/CBP-associated factor (PCAF/KAT2B).

As used herein, the term “HAT inhibitor” refers to a molecule that at least partially blocks, inhibits, reduces or in any way perturbs the deposition of an acetyl group onto a lysine residue in a histone tail. A HAT inhibitor need not completely abolish HAT activity, but rather needs to only inhibit or decrease the activity. In some embodiments, the HAT inhibitor is a direct inhibitor. As used herein, a “direct HAT inhibitor” is a molecule that directly interacts with the HAT and inhibits its acetyltransferase function. In some embodiments, acetyltransferase function is lysine acetyltransferase function. In some embodiments, the HAT inhibitor is a lysine acetyltransferase inhibitor. In some embodiments, the histone is H2A. In some embodiments, the histone is H2B. In some embodiments, the histone is H3. In some embodiments, the histone is H4. In some embodiments, the HAT inhibitor is not a P300/CBP-associated factor (PCAF/KAT2B) inhibitor. In some embodiments, the HAT inhibitor does not directly inhibit P300/CBP-associated factor (PCAF/KAT2B). In some embodiments, HAT inhibition comprises an increase in heterochromatin. In some embodiments, HAT inhibition comprises a decrease in cuchromatin. In some embodiments, HAT inhibition comprises RT inhibition. It will be understood by a skilled artisan that histone acetylation and cuchromatin formation is upstream of reverse transcription of repeat elements and retrotransposons. Thus, HAT inhibition will generate heterochromatin which will inhibit RT of transposable elements, but RT inhibition will have no effect on chromatin status. HAT inhibitors are well known in the art and any may be used as part of the invention. Further, assays for assessing HAT activity are well known and the determination of whether a molecule is a HAT inhibitor can be easily made by a skilled artisan by performing the HAT assay in the presence of the molecule. In some embodiments, the method comprises determining if a compound is a HAT inhibitor. In some embodiments, the method comprises assaying if a compound is a HAT inhibitor. In some embodiments, the method comprises selecting a compound that is a HAT inhibitor.

In some embodiments, the HAT inhibitor is a small molecule inhibitor. In some embodiments, the HAT inhibitor is an inhibitory nucleic acid molecule. In some embodiments, the nucleic acid molecule is an RNA. In some embodiments, the nucleic acid molecule is an antisense oligonucleotide. In some embodiments, the HAT inhibitor is selected form the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, Epigallocatechin gallate (EGCG), anacardic acid, Gambogic acid, Delphinidin, Remodelin, butyrolactone 3 (MB-3), NK13650A, Taxodionc (NP-15), Δ12-prostaglandin J2 (PGJ2), Plumbagin (RTK1), NU9056, DC-M01-7, CCT077791,NSC694614, PU141, C646, TH1834, L002, WM-8014, DC-G16-11, and derivatives thereof. In some embodiments, the HAT inhibitor is selected form the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, Epigallocatechin gallate (EGCG), Delphinidin, Remodelin, butyrolactone 3 (MB-3), NK13650A, Taxodione (NP-15), Δ12-prostaglandin J2 (PGJ2), Plumbagin (RTK1), NU9056, DC-M01-7, CCT077791, NSC694614, PU141, C646, TH1834, L002, WM-8014, DC-G16-11, and derivatives thereof. In some embodiments, the HAT inhibitor is selected form the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, Delphinidin, Remodelin, butyrolactone 3 (MB-3), NK13650A, Taxodione (NP-15), Δ12-prostaglandin J2 (PGJ2), Plumbagin (RTK1), NU9056, DC-M01-7, CCT077791, NSC694614, PU141, C646, TH1834, L002, WM-8014, DC-G16-11, and derivatives thereof. In some embodiments, the HAT inhibitor is a naturally occurring molecule. In some embodiments, the HAT inhibitor is an artificial molecule. In some embodiments, the HAT inhibitor is selected from curcumin and garcinol. In some embodiments, the HAT inhibitor is curcumin. In some embodiments, the HAT inhibitor is garcinol. In some embodiments, the HAT inhibitor is plumbagin. In some embodiments, the HAT inhibitor is selected form the group consisting of: Curcumin, Garcinol, and plumbagin. In some embodiments, the HAT inhibitor is selected form the group consisting of: Curcumin and garcinol. In some embodiments, the HAT inhibitor is selected form the group consisting of: Curcumin and plumbagin.

In some embodiments, the HAT inhibitor is not an acid. In some embodiments, the HAT inhibitor is not Epigallocatechin gallate (EGCG), anacardic acid, or Gambogic acid. EGCG is also acidic and along with anacardic acid and gambogic acid will lower the pH of media to which it is added. In some embodiments, the medium comprises neutral pH. In some embodiments, the medium comprises neutral pH before addition of the HAT inhibitor or RT inhibitor. In some embodiments, the medium comprises neutral pH after addition of the HAT inhibitor. In some embodiments, neutral pH is a pH from about 6.8-7.8. In some embodiments, a neutral pH is a pH from about 6.9-7.6. In some embodiments, a neutral pH is a pH of about 7. In some embodiments, a neutral pH is a pH of about 7.3. In some embodiments, a neutral pH is a pH of about 7.4. In some embodiments, a neutral pH is a physiological pH.

In some embodiments, the medium after addition of the HAT inhibitor or the RT inhibitor does not comprise an acidic pH. In some embodiments, an acidic pH is a pH below 7, 6.9, 6.8, 6.6, 6.5, 6.4, 6.3, 6.2, 6.1, 6, 5.9, 5.8, 5.7, 5.6, 5.5, 5.4, 5.3, 5.2, 5.1 or 5. Each possibility represents a separate embodiment of the invention. In some embodiments, an acidic pH is a pH below 6.9. In some embodiments, an acidic pH is a pH below 6.8. In some embodiments, an acidic pH is a pH below 6. In some embodiments, the medium after addition of the HAT inhibitor or the RT inhibitor comprises a pH above 5, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7. Each possibility represents a separate embodiment of the invention. In some embodiments, the medium after addition of the HAT inhibitor or the RT inhibitor comprises a pH above 6. In some embodiments, the medium after addition of the HAT inhibitor or the RT inhibitor comprises a pH above 6.8. In some embodiments, the medium after addition of the HAT inhibitor or the RT inhibitor comprises a pH above 6.9.

In some embodiments, the medium is pH buffered. In some embodiments, the medium before addition of the HAT inhibitor or RT inhibitor is sufficiently buffered such that addition of the HAT inhibitor or the RT inhibitor does not render the medium acidic. In some embodiments, the medium before addition of the HAT inhibitor or RT inhibitor is sufficiently buffered such that addition of the HAT inhibitor or the RT inhibitor does not reduce the pH below 6.8. In some embodiments, the medium before addition of the HAT inhibitor or RT inhibitor is sufficiently buffered such that addition of the HAT inhibitor or the RT inhibitor does not reduce the pH below 6.9. In some embodiments, the medium before addition of the HAT inhibitor or RT inhibitor is sufficiently buffered such that addition of the HAT inhibitor or the RT inhibitor does not reduce the pH below 6.

In some embodiments, the HAT inhibitor is present at a concentration sufficient to increase constitutive heterochromatin levels in the oocyte. In some embodiments, increase is significantly increase. In some embodiments, significantly is statistically significantly. In some embodiments, increase is at least by a predetermined threshold. In some embodiments, the threshold is an increase of 10, 20, 25, 30, 35, 40, 45, 50, 55, 60, 65, 70, 75, 80, 85, 90, 95 or 100%. Each possibility represents a separate embodiment of the invention. In some embodiments, the threshold is an increase of 25%. In some embodiments, the threshold is an increase of 50%.

In some embodiments, the HAT inhibitor is present at a concentration sufficient to induce maturation. In some embodiments, induce maturation is increase maturation. In some embodiments, the increase is as compared to culturing in media devoid of the HAT inhibitor. In some embodiments, the increase is an increase of at least 10, 20, 25, 30, 35, 40, 45, 50, 55, 60, 65, 70, 75, 80, 85, 90, 95 or 100%. Each possibility represents a separate embodiment of the invention. In some embodiments, the increase is at least a 25% increase. In some embodiments, the increase is at least a 50% increase. In some embodiments, the increase is at least a doubling.

In some embodiments, maturation comprises first polar body extrusion. In some embodiments, maturation comprises chromosome division. In some embodiments, chromosome division is chromosome segregation. In some embodiments, chromosome division is proper or functional chromosome division. In some embodiments, functional is division that produces an oocyte that can be fertilized. In some embodiments, inducing maturation is inducing maturation in at least 20, 25, 30, 35, 40, 45, 50, 55, 60, 65, 70, 75, 80, 85, 90, 95 or 99% of oocytes exposed to the HAT inhibitor. Each possibility represents a separate embodiment of the invention. In some embodiments, inducing maturation is inducing maturation in at least 30% of oocytes. In some embodiments, inducing maturation is inducing maturation in at least 40% of oocytes.

In some embodiments, the curcumin is present in the culture or media at a concentration of about 1-50, 1-40, 1-30, 1-20, 1-15, 1-10, 2-50, 2-40, 2-30, 2-20, 2-25, 2-10, 5-50, 5-40, 5-30, 5-20, 5-15, 5-10, 7-50, 7-40, 7-30, 7-20, 7-15, 7-10, 10-50, 10-40, 10-30, 10-20 or 10-15 μM. Each possibility represents a separate embodiment of the invention. In some embodiments, the curcumin is present in the culture or media at a concentration of about 5-20 μM. In some embodiments, the curcumin is present in the culture or media at a concentration of about 10 μM.

In some embodiments, the plumbagin is present in the culture or media at a concentration of about 0.1-50, 0.1-40, 0.1-30, 0.1-20, 0.1-15, 0.1-10, 0.1-5, 0.1-2.5, 0.5-50, 0.5-40, 0.5-30, 0.5-20, 0.5-15, 0.5-10, 0.5-5, 0.5-2.5, 1-50, 1-40, 1-30, 1-20, 1-15, 1-10, 1-5, 1-2.5, 2-50, 2-40, 2-30, 2-20, 2-25, 2-10, 2-5, 2-2.5, 2.5-50, 2.5-40, 2.5-30, 2.5-20, 2.5-15, 2.5-10, or 2.5-5 μM. Each possibility represents a separate embodiment of the invention. In some embodiments, the plumbagin is present in the culture or media at a concentration of about 0.5-5 μM. In some embodiments, the plumbagin is present in the culture or media at a concentration of about 1-2.5 μM.

As used herein, the term “RT inhibitor” refers to a molecule that at least partially blocks, inhibits, reduces or in any way perturbs the reverse transcription or the action of a reverse transcriptase. A RT inhibitor need not completely abolish RT activity, but rather needs to only inhibit or decrease the activity. In some embodiments, the RT inhibitor is a direct inhibitor. As used herein, a “direct RT inhibitor” is a molecule that directly interacts with the RT and inhibits its reverse transcriptase function. In some embodiments, RT inhibition comprises a reduction in transcription of retrotransposons. In some embodiments, RT inhibition comprises a reduction in retrotransposon expression. In some embodiments, RT inhibition comprises a reduction in retrotransposon hoping. In some embodiments, RT inhibition comprises a reduction in retrotransposon silencing. RT inhibitors are well known in the art and any may be used as part of the invention. Further, assays for assessing RT activity are well known and the determination of whether a molecule is a RT inhibitor can be easily made by a skilled artisan by performing the RT assay in the presence of the molecule. In some embodiments, the method comprises determining if a compound is an RT inhibitor. In some embodiments, the method comprises assaying if a compound is an RT inhibitor. In some embodiments, the method comprises selecting a compound that is an RT inhibitor.

In some embodiments, the RT inhibitor is present at a concentration sufficient to induce maturation. In some embodiments, induce maturation is increase maturation. In some embodiments, the increase is as compared to culturing in media devoid of the RT inhibitor. In some embodiments, the increase is an increase of at least 10, 20, 25, 30, 35, 40, 45, 50, 55, 60, 65, 70, 75, 80, 85, 90, 95 or 100%. Each possibility represents a separate embodiment of the invention. In some embodiments, the increase is at least a 25% increase. In some embodiments, the increase is at least a 50% increase. In some embodiments, the increase is at least a doubling.

In some embodiments, maturation comprises first polar body extrusion. In some embodiments, maturation comprises chromosome division. In some embodiments, chromosome division is chromosome segregation. In some embodiments, chromosome division is proper or functional chromosome division. In some embodiments, functional is division that produces an oocyte that can be fertilized. In some embodiments, inducing maturation is inducing maturation in at least 20, 25, 30, 35, 40, 45, 50, 55, 60, 65, 70, 75, 80, 85, 90, 95 or 99% of oocytes exposed to the RT inhibitor. Each possibility represents a separate embodiment of the invention. In some embodiments, inducing maturation is inducing maturation in at least 30% of oocytes. In some embodiments, inducing maturation is inducing maturation in at least 40% of oocytes.

In some embodiments, the RT inhibitor is a small molecule inhibitor. In some embodiments, the RT inhibitor is an inhibitory nucleic acid molecule. In some embodiments, the nucleic acid molecule is an RNA. In some embodiments, the nucleic acid molecule is an antisense oligonucleotide. In some embodiments, the RT inhibitor is selected form the group consisting of: zidovudine, didanosine, zalcitabine, stavudine, lamivudine, abacavir, emtricitabine, entecavir, truvada, azvudine, tenofovir, adefovir, efavirenz, nevirapine, delavirdine, etravirine, rilpivirine, doravirine, islatravir, and derivatives thereof. In some embodiments, the RT inhibitor is a naturally occurring molecule. In some embodiments, the RT inhibitor is an artificial molecule. In some embodiments, the RT inhibitor is an antiviral agent. In some embodiments, the RT inhibitor is zidovudine (azidothymidine, AZT). In some embodiments, the RT inhibitor is AZT. In some embodiments, the RT inhibitor is lamivudine. In some embodiments, lamivudine is 3TC lamivudine. In some embodiments, the RT inhibitor is selected from AZT and lamivudine.

In some embodiments, the HAT inhibitor is present at a concentration sufficient to decrease retrotransposon expression in the oocyte. In some embodiments, the RT inhibitor is present at a concentration sufficient to decrease retrotransposon expression in the oocyte. In some embodiments, decrease is significantly decrease. In some embodiments, significantly is statistically significantly. In some embodiments, decrease is at least by a predetermined threshold. In some embodiments, the threshold is a decrease of 5, 10, 15, 20, 25, 30, 35, 40, 45, 50, 55, 60, 65, 70, 75, 80, 85, 90, 95, 97 or 100%. Each possibility represents a separate embodiment of the invention. In some embodiments, the threshold is a decrease of 25%. In some embodiments, the threshold is a decrease of 50%.

In some embodiments, the AZT is present in the culture or media at a concentration of about 0.1-10, 0.1-7, 0.1-5, 0.1-3, 0.1-2, 0.1-1, 0.2-10, 0.2-7, 0.2-5, 0.2-3, 0.2-25, 0.2-1, 0.5-10, 0.5-7, 0.5-5, 0.5-3, 0.5-2, 0.5-1, 0.7-10, 0.7-7, 0.7-5, 0.7-3, 0.7-2, 0.7-1, 1-10, 1-7, 1-5, 1-3 or 1-2 μM. Each possibility represents a separate embodiment of the invention. In some embodiments, the AZT is present in the culture or media at a concentration of about 0.5-5 μM. In some embodiments, the AZT is present in the culture or media at a concentration of about 1 μM.

In some embodiments, the lamivudine is present in the culture or media at a concentration of about 0.1-10, 0.1-7, 0.1-5, 0.1-3, 0.1-2, 0.1-1, 0.2-10, 0.2-7, 0.2-5, 0.2-3, 0.2-2, 0.2-1, 0.5-10, 0.5-7, 0.5-5, 0.5-3, 0.5-2, 0.5-1, 0.7-10, 0.7-7, 0.7-5, 0.7-3, 0.7-2, 0.7-1, 1-10, 1-7, 1-5, 1-3, 1-2, 2-10, 2-7, 2-5, 2-3, 3-10, 3-7, 3-5, 5-10, or 5-7 μM. Each possibility represents a separate embodiment of the invention. In some embodiments, the lamivudine is present in the culture or media at a concentration of about 1-10 μM. In some embodiments, the lamivudine is present in the culture or media at a concentration of about 5 μM.

The term “derivative” as used herein, refers to any compound that is based off a known inhibitor and retains the inhibitory function. Assays for testing HAT inhibition and RT inhibition are well known in the art and so a derivative can be easily tested to see if it retains this function. In some embodiments, the method comprises testing if a molecule possesses HAT or RT inhibitory function.

In some embodiments, the medium comprises both a HAT inhibitor and an RT inhibitor. In some embodiments, the medium comprises a plurality of HAT inhibitors. In some embodiments, the medium comprises a plurality of RTT inhibitors. In some embodiments, the culture media comprises curcumin and AZT. In some embodiments, the culture media comprises garcinol and AZT. In some embodiments, the culture media comprises plumbagin and AZT. In some embodiments, the culture media comprises curcumin and lamivudine. In some embodiments, the culture media comprises garcinol and lamivudine. In some embodiments, the culture media comprises plumbagin and lamivudine. In some embodiments, the culture medium is a culture medium of the invention.

In some embodiments, the culturing is for a time sufficient for maturation of the oocyte. In some embodiments, a time sufficient for maturation is a time sufficient for the oocyte to longer be immature. In some embodiments, the culturing is for a time sufficient for first polar body extrusion. In some embodiments, the culturing is until first polar body extrusion. In some embodiments, the time is at least 12, 15, 16, 18, 20, 22 or 25 hours. Each possibility represents a separate embodiment of the invention. In some embodiments, the time is at least 18 hours. In some embodiments, the time is at most 60, 65, 70, 72, 74, 75, or 80 hours. Each possibility represents a separate embodiment of the invention. In some embodiments, the time is at most 72 hours. In some embodiments, the time is about 18-48, 18-54, 18-60, 18-66, 18-72, 24-48, 24-54, 24-60, 24-66, 24-72, 36-48, 36-54, 36-60, 36-66, 36-72, 48-54, 48-60, 48-66, or 48-72 hours. Each possibility represents a separate embodiment of the invention. In some embodiments, the time is about 18-72 hours. In some embodiments, the time is about 48 hours.

In some embodiments, the culturing further comprises the addition of a meiosis inhibitor. In some embodiments, the meiosis inhibitor is a meiosis entry inhibitor. In some embodiments, the method comprises a preculturing before the culturing, wherein the preculturing comprises a meiosis inhibitor. In some embodiments, the culturing is a biphasic culturing comprising a first culturing comprising a meiosis inhibitor and a second culturing comprising the HAT inhibitor or RT inhibitor. In some embodiments, the media is replaced between the preculturing and the culturing. In some embodiments, the media is replaced between the first culturing and the second culturing. In some embodiments, the oocyte is washed between the two phases of culturing or between the preculturing and culturing.

Meiosis inhibitors are well known in the art and any such inhibitor can be used in the preculturing/first culturing. These include various CDK inhibitors, oocyte maturation inhibitor (OMI), WEE2 inhibitors, phosphodiesterase inhibitors, adenosine receptor antagonists and numerous others. In some embodiments, the meiosis inhibitor is C-type natriuretic peptide (CNP). In some embodiments, the meiosis inhibitor is Isobutylmethylxanthine, 1-Methyl-3-Isobutylxanthine (IBMX) In some embodiments, the meiosis inhibitor is present at a concentration sufficient to inhibit entry into meiosis.

In some embodiments, the method elevates as compared to IVM performed in the absence of said HAT inhibitor. In some embodiments, the method elevates as compared to IVM performed in the absence of said RT inhibitor. In some embodiments, the method elevates as compared to IVM performed in the absence of said HAT inhibitor and said RT inhibitor. In some embodiments, elevates is increases. In some embodiments, the method further comprises selecting a mature oocyte. In some embodiments, the method further comprises selecting an oocyte with increased heterochromatin. In some embodiments, the method further comprises selecting an oocyte with decreased retrotransposon expression.

### Culture Media

By another aspect, there is provided a medium comprising at least one gonadotropin and at least one HAT inhibitor or RT inhibitor.

By another aspect, there is provided a kit comprising:
a. medium;
b. at least one gonadotropin; and
c. at least one HAT inhibitor or RT inhibitor.

In some embodiments, the medium is culture medium. In some embodiments, the medium is cell culture medium. In some embodiments, the medium is tissue culture medium. In some embodiments, the medium is IVM medium. In some embodiments, the medium is sterile. In some embodiments, the medium is chemically defined. In some embodiments, the medium is devoid of animal product. In some embodiments, the animal is a non-human.

In some embodiments, the gonadotropin is mammalian gonadotropin. In some embodiments, the mammal is a human. In some embodiments, the mammal is a veterinary animal. In some embodiments, the mammal is a domesticated animal. In some embodiments, the mammal is a livestock animal. In some embodiments, the veterinary animal is selected from, a cat, a dog, a horse, a cow, a pig, a sheep and a goat. In some embodiments, the gonadotropin is a human gonadotropin. In some embodiments, the gonadotropin is LH. In some embodiments, the gonadotropin is FSH. In some embodiments, the gonadotropin is LH and FSH.

In some embodiments, the medium is for use in IVM. In some embodiments, the kit is for use in IVM. In some embodiments, IVM is IVM of an oocyte. In some embodiments, the medium is for use in IVF. In some embodiments, the kit is for use in IVF. In some embodiments, IVF is IVF of an oocyte. In some embodiments, the oocyte is a mammalian oocyte. In some embodiments, the oocyte is a human oocyte.

### Patient Selection

By another aspect, there is provided a method of determining suitability of a subject to be treated by a method of the invention, the method comprising:
a. receiving from a female subject at least one oocyte; and
b. measuring heterochromatin in the oocyte, wherein a heterochromatin level below a predetermined threshold indicates the subject is suitable for IVM by a method of the invention;
thereby determining the suitability of a subject to be treated by a method of the invention.

In some embodiments, the oocyte is an immature oocyte. In some embodiments, the oocyte is a geminal vesicle (GV) oocyte. In some embodiments, the oocyte is a GV-arrested oocyte. In some embodiments, the oocyte was harvested for IVF. In some embodiments, the method further comprises harvesting the oocyte from a female. In some embodiments, subject is a human. In some embodiments, the received oocyte was found immature. In some embodiments, the received oocyte was immature. In some embodiments, immature is not sufficiently mature. In some embodiments, the received oocyte was unsuitable for IVF. In some embodiments, the subject has undergone at least one unsuccessful round of IVF. In some embodiments, unsuccessful is a round of IVF that does not produce implantation. In some embodiments, implantation is embryo implantation. In some embodiments, unsuccessful is a round of IVF that does not produce pregnancy. In some embodiments, unsuccessful is a round of IVF that does not produce a fetus. In some embodiments, unsuccessful is a round of IVF that does not produce a birth. In some embodiments, unsuccessful is a round of IVF that does not produce a baby. A skilled artisan in the field of IVF can determine if an oocyte is suitable for IVF. Unused oocytes are suitable for use in the diagnostic method. In some embodiments, heterochromatin levels above the predetermined threshold indicate the subject is suitable for another round of IVF. In some embodiments, heterochromatin levels below the predetermined threshold indicate the subject is unsuitable for another round of IVF. In some embodiments, heterochromatin levels below the predetermined threshold indicate the subject is suitable for a method of the invention. In some embodiments, heterochromatin levels below the predetermined threshold indicate the subject is suitable for a method of the invention followed by IVF. In some embodiments, the method further comprises performing the IVM method of the invention on a suitable subject. In some embodiments, the method further comprises performing another round of IVF on a subject with chromatin levels above the predetermined threshold.

Assays for measuring heterochromatin are well known in the art and any such assay may be used. Methods include those disclosed hereinbelow, western blot, immunostaining, chromatin immunoprecipitation, sequencing, methyl-sequencing, bisulfate sequencing and the like. In some embodiments, measuring heterochromatin comprises measuring histone 3 lysine 9 (H3K9) methylation. In some embodiments, methylation comprises monomethylation. In some embodiments, methylation comprises dimethylation. In some embodiments, methylation comprises trimethylation. In some embodiments, measuring heterochromatin comprises measuring H3K9me3. In some embodiments, measuring heterochromatin comprises measuring H4K20 methylation. In some embodiments, measuring heterochromatin comprises measuring H4K20me3. In some embodiments, measuring heterochromatin comprises measuring heterochromatin protein 1 (HP1). In some embodiments, measuring HP1 comprises measuring HP1 levels. In some embodiments, levels are mRNA levels. In some embodiments, levels are protein levels. In some embodiments, measuring heterochromatin comprises measuring methylcytosine. In some embodiments, methylcytosine is 5 methylcytosine. In some embodiments, methylcytosine is methylcytosine levels.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Animals: RCC-C57BL/6JHsd female mice were used for the experiment. For the young group, we used 7-11-week-old mice, and for the old group we used 8-10-month-old mice. Mice were used after an acclimation period of at least 3 days after shipment. The experiment was approved by the institutional ethics committee.

Mouse oocyte in vitro maturation: After Euthanasia, ovaries were collected and dissected in L-15 medium (011151A) and supplemented with 200 um IBMX (17018) to prevent meiotic progression. Prophase I arrested oocytes were identified by visualization of the typical germinal vesicle and collected under a binocular using a Stripper (MD-MXL3-STR-CGR). After collection, oocytes were transferred into a-MEM medium (22561021) supplemented with IBMX covered with Mineral oil (M8410-11) to prevent evaporation, for recovery time of 25 min at 37° in a 5% CO2 incubator, and then washed in IBMX free a-MEM medium to initiate meiosis. The oocytes were incubated in a-MEM under oil in the incubator for 6 hours for prophase I or incubated for 19 hours for metaphase II. To assess maturation rates, oocytes were incubated for 19 hours after release and then fixed and stained by Hoechst to examine the entry into the second meiotic division. See below amendments for drug treatments.

Mouse oocytes collection for in situ immunofluorescence: After Euthanasia, ovaries were collected and dissected in M2 medium (M7167). Oocytes were collected from pool of 3 animals at least (numbers of each experiment are noted in legend). Prophase I arrested oocytes were collected under a binocular using a Stripper and washed in hyaluronidase (H4272-30 MG) to remove granulosa cells and acidic Tyrode's Solution (T1788) to remove the Zona Pellucida. The oocytes were fixed using PFA 4% (15710) for 20 min, and then quenched in PBS supplemented with 10 mM glycine and 1% BSA.

Chromosome spread and immunofluorescence: Prophase I arrested oocytes were collected from ovaries as above and incubated for 6 hours to reach the first prophase or for 17 hours to reach second meiosis prophase as described above. Matured oocytes were washed in M2 medium and then in acidic Tyrode's Solution to remove the Zona Pellucida. 15 min before the desired time for chromosome spread, oocytes were kept in hypotonic solution, composed of FBS (F7524) diluted in DDW in 1:1 ratio. Oocytes were then spread in spreading solution (1% PFA buffered to 9.2 pH, supplemented with 0.15% Triton X-100and 0.03% DDT) on Superfrost plus slides (32090003). To assess ploidy, prophase II spreads were stained with Hoechst to visualize chromatin and CREST to visualize the centromeres, and the imaged chromosome were manually counted.

In situ immunofluorescence: Permeabilization was performed using 0.1% Triton X-100. Cells were cultured in PBS contains 5% BSA for blocking, and first and secondary antibodies were diluted in 0.1 Tween 20 (P9416)/PBS containing 5% BSA. The immune-stained cells were mounted in Vectashield (H-1000) mounting medium containing 80 nM of Hoechst (33342) and sealed on a slide using an Imaging spacer (GBL654002).

Mouse ovaries IHF: Ovaries were collected, washed in PBS and fixed with shaking at 4° for 24 hours in 4% PFA. After fixation the ovaries were kept in 30% sucrose with shaking at 4° overnight. The tissue was kept in Tissue-Tek OCT Compound (4583) in −80°.Cryo-sectioning was performed on a Leica CM 1950 Cryostat, and sections were applied on superfrost plus slides. In order to capture most of the oocytes' chromatin, that spans over ˜30 um in the Z plane per cell, every section was taken in 20 um thickness. To avoid having the same oocytes (50-70 um˜) in two different sections on the same slide, every slide contained sections that are at least 80 μm apart. Before staining, the slides were dried overnight at room temperature, and washed extensively in PBS to remove OCT. Sections were cultured in PBS containing 1% FBS and 0.1% Triton X-100 for blocking. All antibodies were diluted in a concentration of 1:100 in blocking solution. Slides were mounted in Vectashield mounting medium containing 80 nM of Hoechst, and sealed with nail polish.

Antibodies: For heterochromatin staining we used antibodies for H3K9me2 (Abcam, ab1220, working dilution 1:400), H3K27me3 (Abcam, ab205728, 1:100) HP1γ (Abcam, ab227478, 1:50). For cuchromatin markers we used antibodies for H3K27ac (Cell signaling, 8173s, 1:100) and H3K4me3 (Cell signaling, 11960s, 1:50). To track retroviral activity and transcription regulation loss we used antibodies for L1-ORF1p (Abcam, ab216324, 1:100), dsRNA (SCICONS, 10020200, 1:500) and Dicer (Abcam, ab167444, 1:200). Meiotic Cohesin was stained with an antibody against REC8 (Abcam, ab 192241 1:200), and DNA damage was assessed using antibodies against RAD51 (Sigma, PC130, 1:200), and γH2Ax (Sigma, 0563625UG, 1:500). GFP expression was measured using anti-GFP antibody (Rockland 600-101-215, 1:250). Centromere visualization was achieved using CREST antibody (INC 15-235-0001, 1:100).

For western blot primary antibodies include; GAPDH (Abcam, ab8245 1:3000), REC8 (Abcam, ab 192241, 1:4000), L1-ORF1p (Abcam, ab216324, 1:1000), Dicer (Abcam, ab167444, 1:1000), and Tubulin (T5168 1:4000) Anti-rabbit and anti-mouse horseradish peroxidase-conjugated secondary antibodies were used (Goat Anti-Rabbit IgG H&L (HRP) ab6721 (1:10,000), and Rabbit Anti-Mouse IgG H&L (HRP) ab6728 (1:10,000) or Goat Anti-Mouse IgG H&L (HRP) ab97040 (1:10,000).

Imaging and quantification: Oocytes were imaged using the Ti-Eclipse Nikon system, with an Andor Zyla nsc05537 camera. Confocal imaging was used when light imaging failed to obtain a clear image, using Yokogawa W1 Spinning Disk on Motorized fluorescent microscope Ti2E, with 2 SCMOS ZYLA cameras or Zeiss LSM710. Every cell was imaged at multiple planes. In order to analyze the images in a quantitatively, a projection of maximum intensity was created. To measure staining intensity, every assessed region was normalized to an equal-size region in the background (outside the area of interest). The intensity score was generated by dividing the intensity in the area of interest by that of the background.

Imaging and quantification: In order to analyze the images quantitatively, a projection of maxi-mum intensity was created. To measure staining intensity, every assessed region was normalized to an equal-size region in the background (outside the area of interest). The intensity score was generated by dividing the intensity in the area of interest by that of the background.

Small RNA sequencing: ˜50 oocytes (number was matched between groups in every experiment) from young and old females (each in triplicate) were collected from ovaries as above and dissected in medium M2, and after the granulosa cells and the Zona Pellucida were removed (as mentioned above), the oocytes were inserted into 1 ml of TRIzol (15596026). RNA extraction was done according to the TRIzol reagent user guide, with extraction in 10 μl of nuclease-free water. Amplification was performed by the SMARTer® smRNA-Seq Kit for Illumina®-12 Rxns, TAKARA 635029. Size selection was performed using Agencourt AMPure XP Beads, by adding 50 ul of sample to 100 ul of beads solution and eluting the DNA bound to the beads (selecting transcripts below 200 bp). Sequencing was performed at the Ein-Kerem campus interdepartmental equipment park on a NextSeq machine.

Trimming and filtering of raw reads: The NextSeq base-calls files were converted to fastq files using the bc12fastq program with default parameters (without trimming or filtering applied at this stage). Raw reads (fastq files) were inspected for quality issues with FastQC. Following that, and according to the SMARTer smRNA-Seq library construction protocol, the first 3 bases of R1 reads were discarded (positions for template switching), the reads were quality-trimmed at both ends, poly-G sequences (NextSeq's no signal), adapter sequences, and poly-A sequences were removed from the 3′ end, and finally low quality reads were filtered out. Cutadapt was used for trimming sequences from the end of reads, with parameters that included using a minimal overlap of 1, allowing for read wildcards, and filtering out reads that became shorter than 15 nt. Final filtering by quality was performed using the fastq_quality_filter program of the FASTX package, with a quality threshold of 20 at 90 percent or more of the read's positions.

Alignment and counting: The processed reads were aligned to the mouse transcriptome and genome with TopHat. The genome version was GRCm38, with annotations from Ensembl release 99. Quantification was done with htseq-count.

Differential expression: Normalization and differential expression analysis were done with the DESeq2 package. Results were corrected for batch effect and further normalized between samples to a control gene expression (the gene Rian (Hatada et al., 2001, PMID: 11481034) Raw data submitted to GEO under accession GSE159789. Sequencing results appear in Wasserzug-Pash et al., “Loss of heterochromatin and retrotransposon silencing as determinants in oocyte aging”, Aging Cell, 2022 March;21(3): e13568, herein incorporated by reference in its entirety.

qRT-PCR: After Euthanasia, mouse ovaries were collected and dissected in M2 medium (M7167) with 200 um IBMX (I7018) to prevent meiotic progression as above. 40 to 60 (number was matched between groups in every experiment) prophase I arrested oocytes were collected and washed in hyaluronidase (H4272-30 MG) to remove granulosa cells, and then washed extensively in M2 until all the granulosa cells were removed from the oocytes. Oocytes were transferred to TRIzol (15596026) and Chloroform solution for RNA precipitation, treated with RNAse free DNAse (E0013-1D3) to remove DNA and then RNA was purified by using Ampure beads (A63881). Reverse transcription was performed using iScript™ Reverse Transcription Supermix for RT-qPCR (1708891) according to the manufacturer's recommendations. iTaqTM Universal SYBR® Green Supermix (1725124) was used for amplification reactions. Quantitive PCR measurements were taken using a CFX96 C1000 BioRad machine.

Mouse oocytes western blot: After Euthanasia, ovaries were collected and dissected in L-15 medium (011151A) supplemented with 200 μm IBMX (I7018) to prevent meiotic progression. 101 prophase I-arrested oocytes were collected from each and washed in hyaluronidase (H4272-30 MG) to remove granulosa cells, and then washed extensively in L-15 until all the granulosa cells were removed from the oocytes. The clean oocytes were then washed in PBS until all the culture media was gone and transferred into RIPA buffer (1% NP-40, 0.1% SDS, 50 mM Tris, 150 mM NaCl, 0.5% sodium deoxycholate, 1 mM EDTA, 1X complete™ Protease Inhibitor Cocktail 11836145001), and kept on ice. Protein extraction was performed by boiling the lysate in Laemmli buffer (1610747) 335 mM 2-mercptoethanol. The lysates were fractionated by 10% acrylamide SDS gel under reducing conditions and transferred to a nitrocellulose membrane (Millipore) using a transfer apparatus according to the manufacturer's protocol (Bio-Rad). Blots were developed with an ECL system according to the manufacturer's protocol (Bio-Rad). Results were collected using ChemiDoc XRS+System from Bio-Rad. After Euthanasia, ovaries were collected and dissected in L-15 medium (011151A) supplemented with 200 um IBMX (I7018) to prevent meiotic progression. 101 prophase I arrested oocytes were collected from each and washed in hyaluronidase (H4272-30 MG) to remove granulosa cells, and then washed extensively in L15 until all the granulosa cells were removed from the oocytes. The clean oocytes were then washed in PBS until all the culture media was gone and transferred into RIPA buffer (1% NP-40, 0.1% SDS, 50 mM Tris, 150 mM NaCl, 0.5% Sodium Deoxycholate, 1 mM EDTA, 1X complete™ Protease Inhibitor Cocktail 11836145001), and kept on ice. Protein extraction was performed by boiling the lysate in Laemmli buffer (1610747) 335 mM 2-mercptoethanol. The lysates were fractionated by 10% acrylamide SDS gel under reducing conditions and transferred to a nitrocellulose membrane (Millipore) using a transfer apparatus according to the manufacturer's protocol (Bio-Rad). Blots were developed with an ECL system according to the manufacturer's protocol (Bio-Rad). Results were collected using ChemiDoc XRS+System from Bio-Rad.

Drug treatments: Ovaries were collected and dissected in L-15 medium supplemented with 200 um IBMX to prevent meiotic progression as described above. Prophase I arrested oocytes were collected using a Stripper as described above. After collection, oocytes were transferred to a-MEM medium supplemented with IBMX +the desired drug covered with Mineral oil to prevent evaporation, for 4 hours at 37° in a 5% CO2 incubator (Since Chaetocin and IBMX have a cross reactivity with each other, the pause was not performed in Chaetocin experiments). After the pause, oocytes were washed in IBMX-free a-MEM that contains the drug to initiate meiosis. 19 hours after oocytes were released from IBMX, oocytes were fixed in PFA, permeabilized, and sealed as described above. Chaetocin is a hazarzous substance. For biosafety reasons, Chaetocin supplemented plates and matched controls were placed in a sealed chamber during incubation time. Drugs in use were: Trichostatin A (TSA) T1952, Chaetocin (sc-200893), SRT1720 (biovision 2772) and Zidovudine (AZT) (PHR1292-1G).

Oocyte electroporation: Ovaries were collected and dissected in L-15 medium supplemented with 200 um IBMX to prevent meiotic progression as described above. Prophse I oocytes were washed with acidic Tyrode's Solution and transferred into EC-002 electroporation cuvettes containing 100 ul of clean L-15 medium for the control group and 50-200 ng/ul, lonza pmaxGFP (DMC00054) or the gene expressing plasmids for the experimental group. Electroporation was done in Nepagene NEPA21 electroporator. The electroporated oocytes were washed briefly and then cultured in IBMX supplemented a-MEM. 24 hours after electroporation oocytes were fixed and stained with reported antibodies.

Human in vitro fertilization (IVF) protocols: Patients were treated with one of two protocols for ovarian stimulation determined by their physician. Long agonist protocol started in the luteal phase of the menstrual cycle, or antagonist protocol started on the second day of the menstrual cycle. For the controlled ovarian hyperstimulation in both protocols either recombinant FSH preparation, recombinant FSH and LH or HMG (human menopausal gonadotrophin) were used. Dosage was determined individually for each patient according to BMI and ovarian reserve parameters. Ovarian response was monitored by serial ultrasound examinations and the evaluation of serum E2 levels, then gonadotropin doses adjustment was done as required. Human chorionic gonadotropin (HCG) was administered when at least 3 leading follicles were 17-18 mm in diameter. Oocyte retrieval was performed 36 hours following HCG administration using transvaginal ultrasound guided approach.

Human oocytes: Human prophase I arrested oocytes that were retrieved during IVF treatment were incubated for 24 h before determination that they remained at this state and did not mature to become fully grown MII oocytes. After informed consent was signed (following IRB approval 0020-16-SZMC) oocytes were treated by piercing of the Zona Pellucida by the embryologists in order to increase permeability. The oocytes were then fixed in 4% PFA the day after retrieval, for 20 min at room temperature and then quenched in PBS supplemented with 10 mM glycine and 1% BSA. Immunofluorescence was performed as described above. Analysis was performed separately for prophase I arrested oocytes, recognized by the typical germinal vesicle, and dispersed chromatin configuration, and for oocytes which arrested after meiotic resumption, recognized by lack of germinal vesicle and chromatin condensation. Oocytes that entered the second meiotic division, recognized by chromatin division and polar body extrusion were excluded from the data.

REC8 antibody specificity: HCT116 human colon carcinoma cell line cells were maintained in Dulbecco's Modified Eagle's medium (DMEM, Sigma), supplemented with 10% fetal bovine serum (FBS), 1% PenStrep (100 U/mL Penicillin and 100 μg/mL Streptomycin) in a 37° C. incubator (5% CO2). The cell lines were authenticated at the Biomedical Core Facility of the Technion, Haifa, Israel. REC8 tagged with Enhanced Green Fluorescent Protein (EGFP), and EGFP only (vector pEGFP-NI, plasmid no. 170528HM8137-6 from BD Biosciences) were used. The QIAprep® Spin miniprep kit by Qiagen (cat-27104, Germany) was used to extract the plasmid. All cells were transfected 24 h after initial plating. Transfections were performed using the PolyJet™ In Vitro DNA Transfection Reagent (cat-SL100688, USA) with a 3:1 (transfection reagent: DNA) ratio. 48 h after transfection, adherent cells were lysed using hot sample buffer (10% glycerol, 50 mmol/L Tris-HCl pH 6.8, 20% SDS, and 5% 2-mercaptoethanol), and western blot analysis was carried out. The lysates were fractionated by SDS PAGE (Criterion™ TGX (Tris-Glycine eXtended) Stain-Free™ precast gels, Bio-Rad) under reducing conditions and transferred to nitrocellulose membrane (Millipore) using a transfer apparatus according to the manufacturer's protocol (Bio-Rad). Blots were developed with an ECL system according to the manufacturer's protocol (Bio-Rad). Results were collected using ChemiDoc XRS+System from Bio-Rad.

Statistical analysis: Statistical analysis was performed using Excel, R and GraphPad prism. To compare between means—if N>30 or if a Shapiro Wilk test returned insignificant the analysis was performed using a parametric test (t test for single comparison and one way Anova for group comparison). Otherwise, a non-parametric test was used (Mann Whitney (MW) for single comparison and Kruskal Wallis (KW) for group comparison). To compare the difference between groups in transcript presence, ratio-paired T test was used. To compare proportions, Z test for two proportions was used, when normality of parameters was confirmed either by N>30 or by N*P>5 for each of the compared groups. Correlation was calculated using Person's correlation coefficient, and significance of the calculated R was determined using F test.

Human IVM protocol: Germinal vesicle (GV) oocytes were subjected to IVM in a Sage medium (A1-rad Medical) supplemented with 0.075 IU/mL luteinizing hormone (LH) and 0.075 IU/mL follicle stimulating hormone (FSH) overnight. HAT and/or RT inhibitors were added to the overnight culture in some experiments. All naked oocytes that were found were transferred for up to 48 hours of incubation in oocyte maturation medium (Sage In-Vitro Fertilization) supplemented with 0.075 IU/mL human menopausal gonadotropin (hMG) (Menopur, Ferring Pharmaceuticals). HAT and/or RT inhibitors were added to the 48-hour culture in some experiments. After 24 hours of incubation, the media was searched for mature oocytes that were immediately frozen. The media were then incubated for another 24 hours and searched again for other mature oocytes.

Modified human IVF protocol: GV oocytes were incubated in oocyte maturation medium (Sage In-Vitro Fertilization) supplemented with 0.075 IU/mL hMG (Menopur, Ferring Pharmaceuticals) and HAT and/or RT inhibitors. All MII Oocytes (either retrieved or obtained through IVM incubation) were held in oocyte holding medium supplemented with HAT and/or RT inhibitors and ICSI was performed. Fertilized two-pronucleate zygotes were cultured individually in 20 mL of cleavage-stage medium (Sage) for 2 or 3 days, and blastocyst culture was also performed using commercially available media (Quinn's Advantage; Sage). All the embryos were cultured at 37° C. under a gas phase of 5% 02, 5% CO2, and 90% N2 with full humidity in water jacket small multigas incubators (Astec). Blastocyst culture, elective vitrification, and subsequent frozen-thawed ET were performed routinely. Embryos that appeared to have good quality were transferred at cleavage stage. Others were cultured to blastocyst.

### Example 1

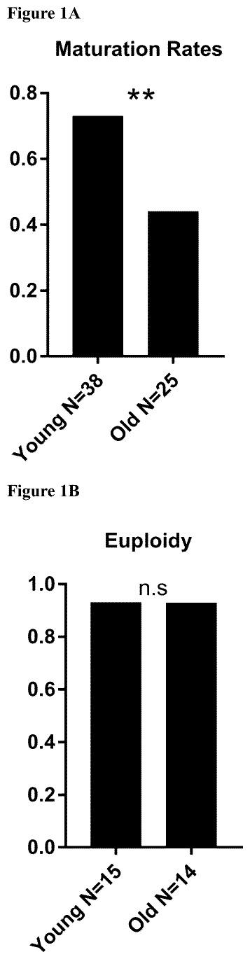

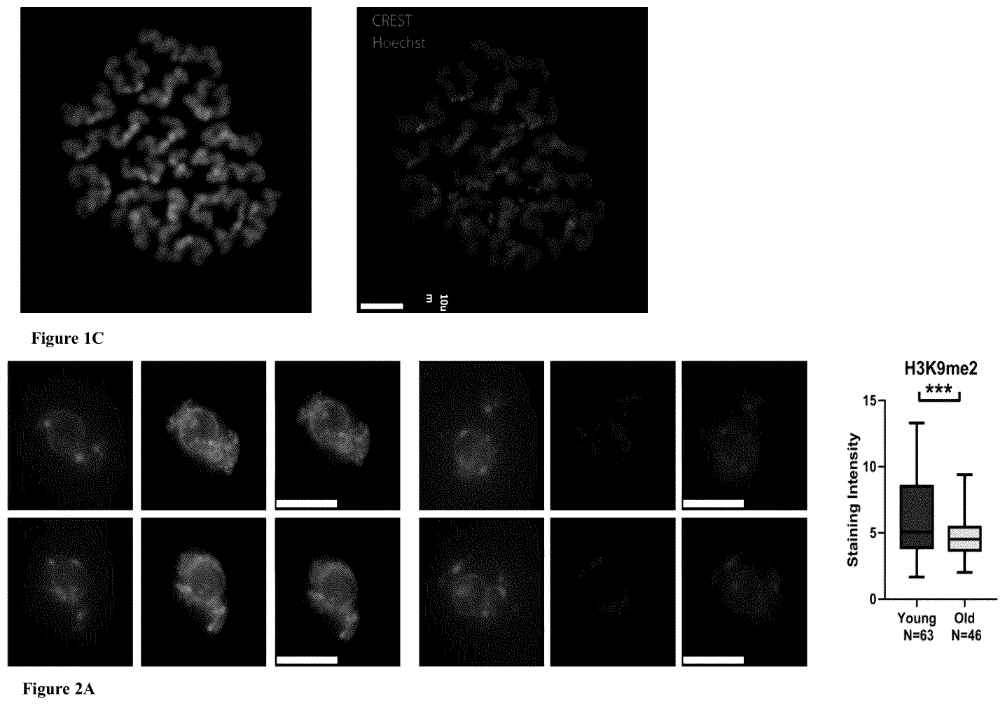

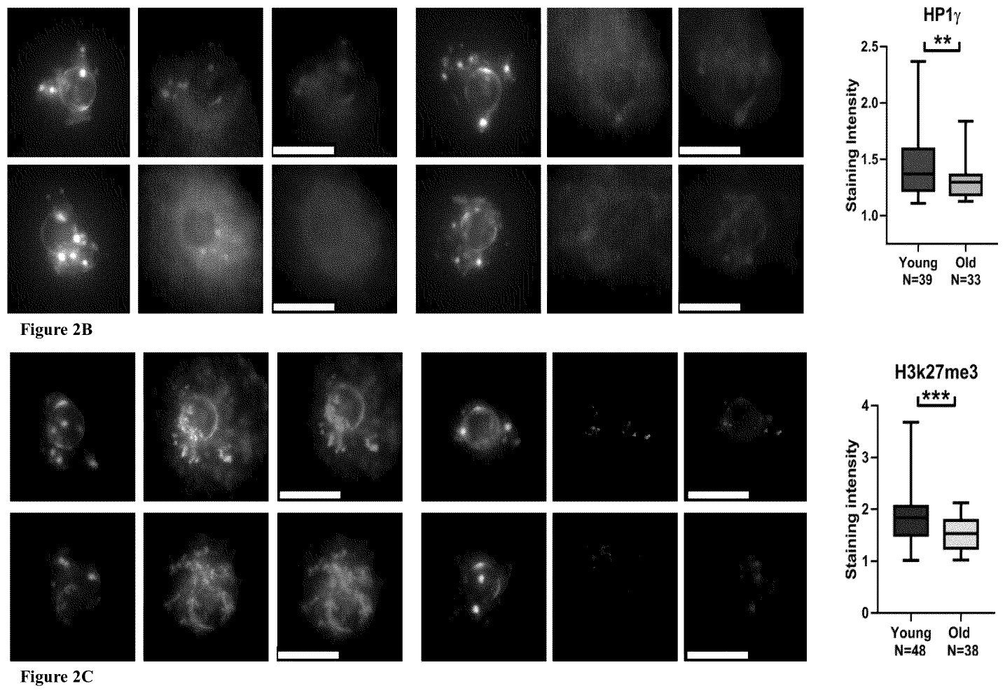

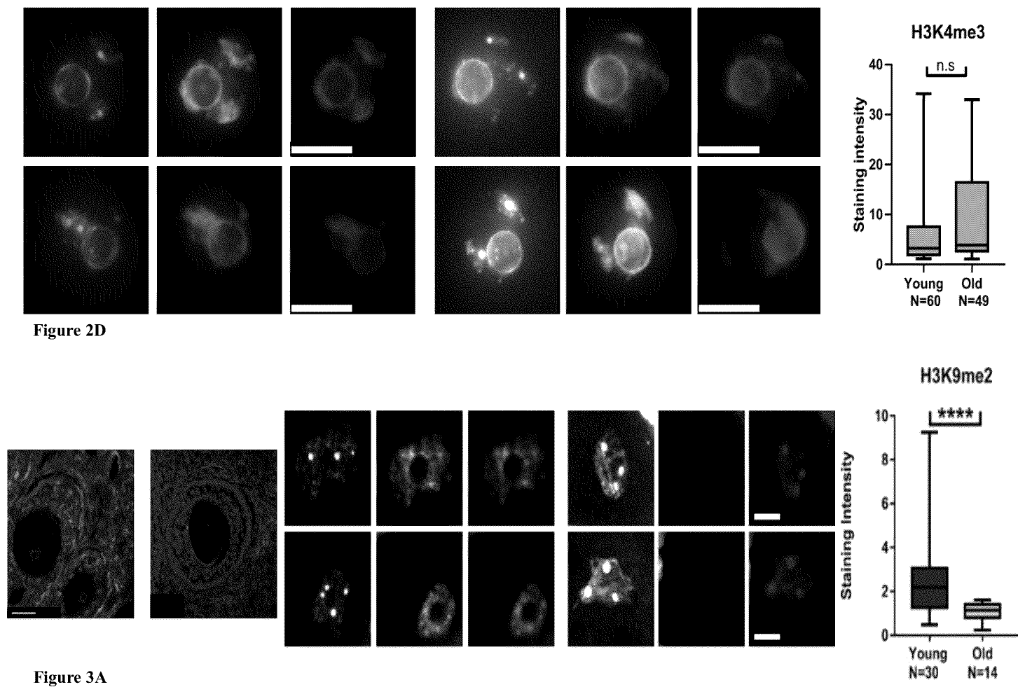

In order to investigate the status of heterochromatin histone marks in oocytes at an age before the onset of aneuploidy, we compared the levels of specific epigenetic markers by immunofluorescence (IF) in prophase I-arrested oocytes of unstimulated (i.e., not super-ovulated) 2-month-old (young) and 9-month-old (old) mouse females. According to previous studies, mouse oocyte aneuploidy at the age of 9 months is low and not significantly different than in young mice. However, other fertility associated traits such as the level of oocyte maturation at 9 months by IVM (in vitro maturation), number of oocytes found in the oviduct after superovulation and fecundity are already markedly reduced. The mouse strain we use (RCC-C57BL/6JHsd, see methods) in the present study shows consistent findings with this decline in maturation rates that were 27% lower in old compared with young mice, and aneuploidy rates, which were identical between both age groups (FIG. 1A-1C). We then proceeded to immunostaining of the oocytes for constitutive heterochromatin markers H3K9me2 and HP1γ. Both markers' levels decrease significantly with age when assessed by in situ immunofluorescent staining (FIG. 2A-2B, see methods for further details). In addition, the level of the facultative heterochromatin mark H3K27me3 also reduces with age when assessed by in situ immunofluorescent staining (FIG. 2C).

Prophase I oocytes in mouse and human have two main chromatin configuration forms, which characterize two developmental stages. The first chromatin configuration in maturing oocytes, is termed non-surrounded nucleolus (NSN), and is characterized by a diffuse chromatin configuration. Gradually, with oocyte growth, chromatin is organized around the nucleolus in a ring-shaped structure, a con-figuration termed surrounded nucleolus (SN). The transition from NSN to SN is accompanied by multiple cellular changes, including comprehensive histone modification changes, and transcription silencing that continues throughout oocyte maturation and early embryogenesis. Interestingly, the defect in heterochromatin modification with age is much more pronounced as the oocyte proceeds to the SN stage—when the oocyte undergoes transcriptional shutdown. However, facultative heterochromatin decrease occurs uniformly in all prophase I arrest stage oocytes (both NSN and SN), showing that some age-related epigenetic defects can already be seen at an early oocyte maturation stage. Importantly, transcription is still active in Prophase I-arrested oocytes as was shown by BrU incorporation into RNA, with complete transcriptional shutdown occurring only later during the oocyte maturation process. This was verified by plasmid electroporation experiments showing expression of eGFP in electroporated Prophase I-arrested oocytes.

To investigate whether the decrease in repressive marks occurs as a result of nucleosome or histone loss and to control for differences in oocyte staining capacity between young and old oocytes, we stained oocytes by in situ immunofluorescence for the active chromatin marks H3K27Ac and H3K4me3. Importantly, no significant difference was detected between young and old oocytes for these two chroma-tin marks (FIG. 2D). These results show that the loss of histone marks is specific to heterochromatin modifications and that no detectable histone loss has occurred in old oocytes.

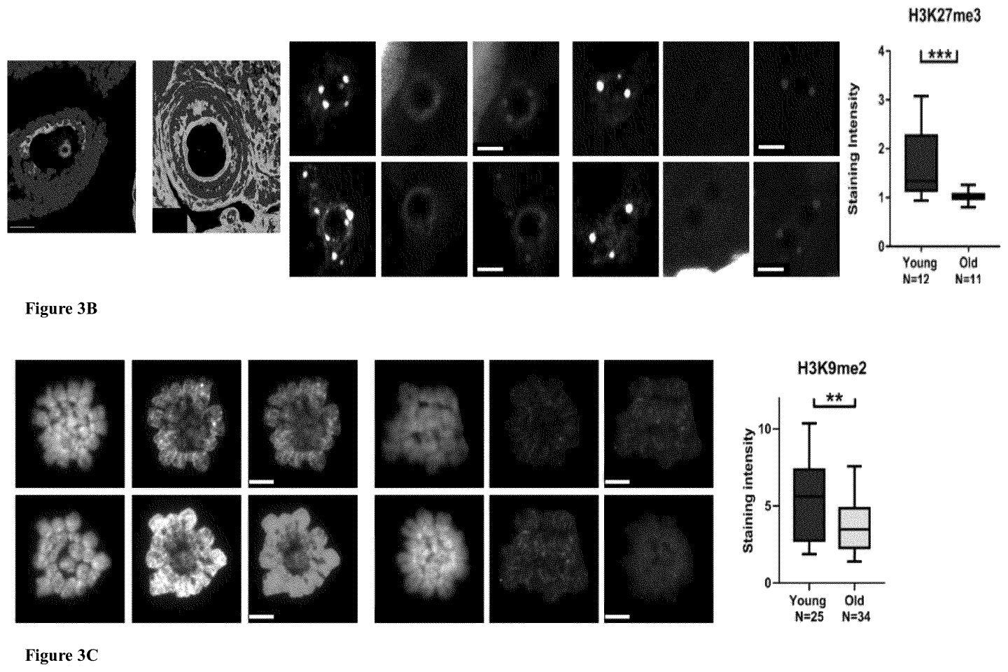

To achieve information about wider range of oocytes population and to avoid possible confounding effects from immunofluorescence staining procedures, we performed IHF on mouse ovaries cryosections. Using this method, we measured heterochromatin levels in a wider variety of oocyte stages and sizes than those achieved by manual collection of oocytes. Heterochromatin loss with age was also apparent in ovary cryosections as demonstrated by the lower level of H3K9me2 and H3K27me3 (FIG. 3A-3B) in the aged ovaries oocytes compared with the young group. This trend was observed in all the oocytes at all sizes. Moreover, after characterizing epigenetic changes in aging non-mature oocytes, we also explored if the epigenetic defect persists till the later stages of oocyte maturation. To do this, we investigated heterochromatin markers of oocytes at metaphase of MI by performing chromosome spreads followed by immunostaining. Spreads were stained for H3K9me2 and for H3K27me3 (FIG. 3C-3D). In these experiments, a significant drop in signal was observed in older oocytes. This result shows that the epigenetic defects that occur in older oocytes persist till the meiotic divisions. Since the spread experiments enable the visualization of the entire chromosome, we also asked whether the decrease in re-pressive chromatin marks occurs on specific loci in the genome, or whether the decrease is uniform along the entire chromosome. No significant pattern of specific preference was found in our analysis. Despite the limited ability to discern specific regions of the genome by microscopy, this result suggests that the decrease in heterochromatin is widespread and includes many regions in the genome.

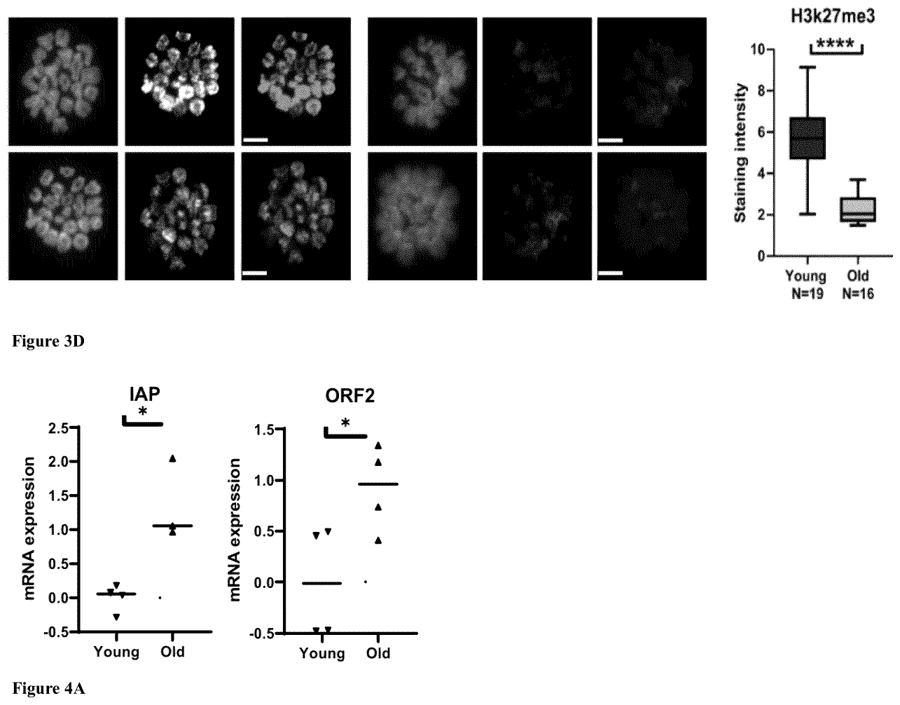

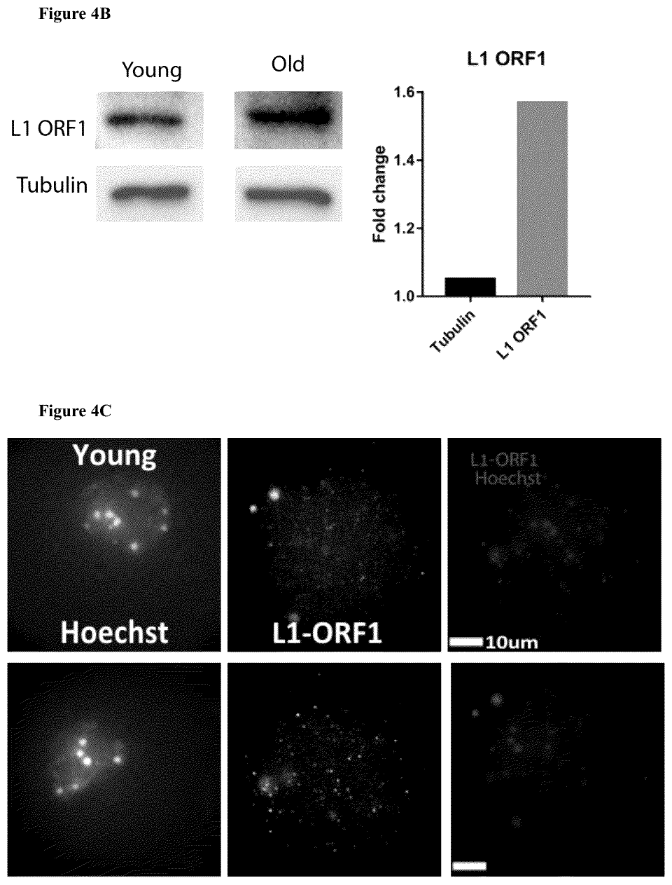

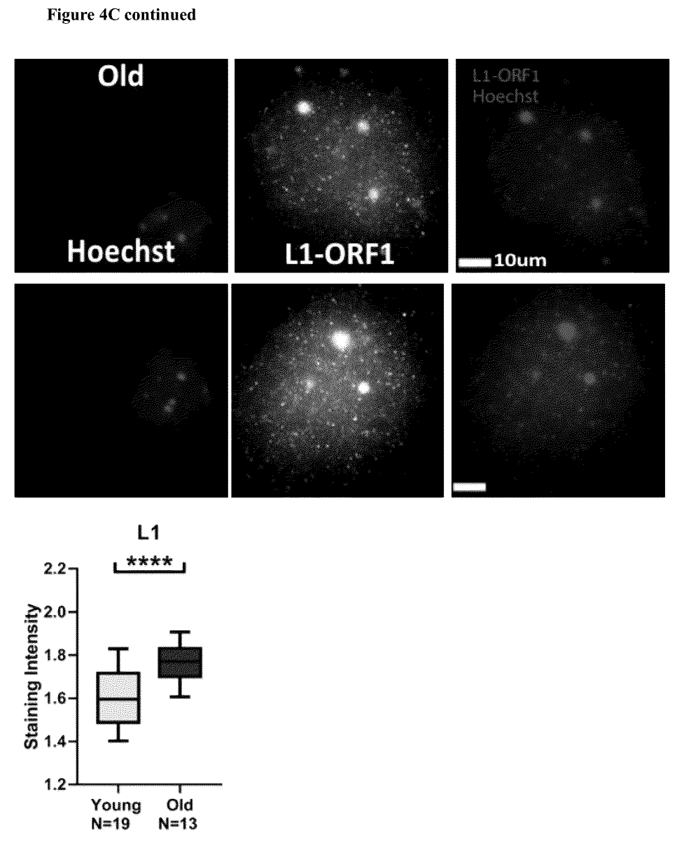

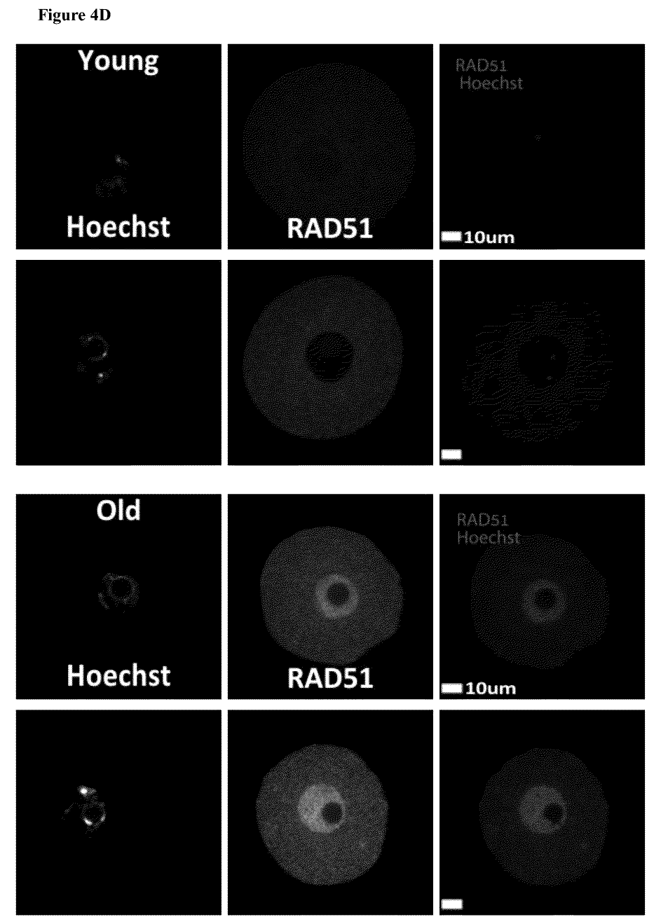

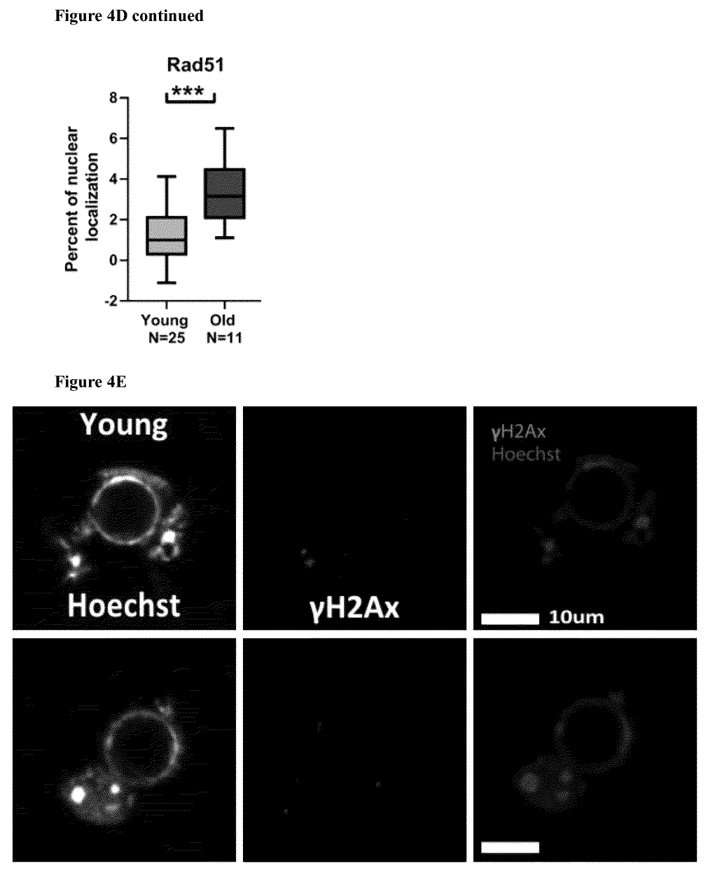

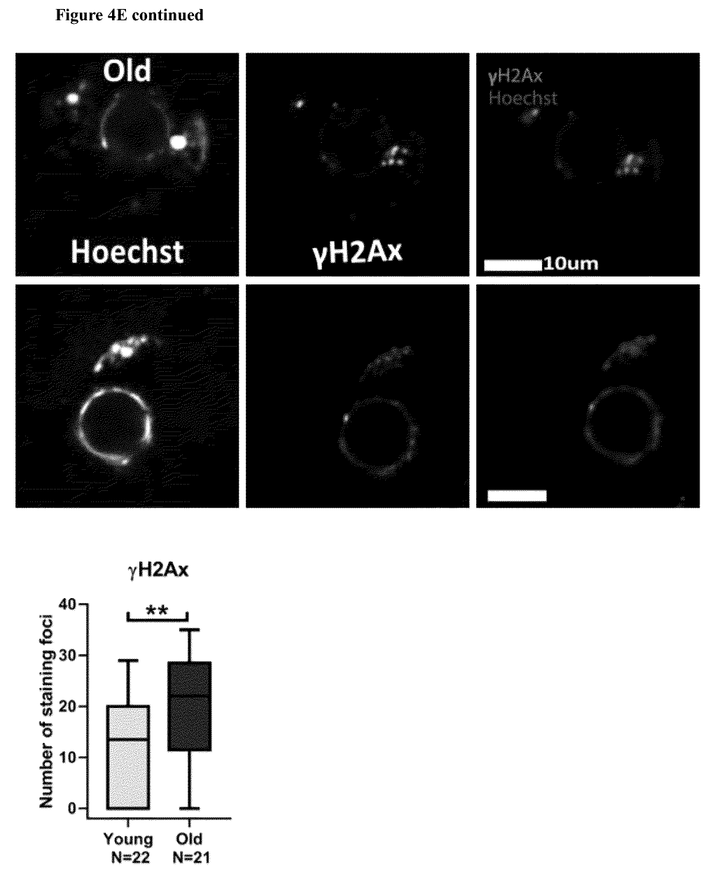

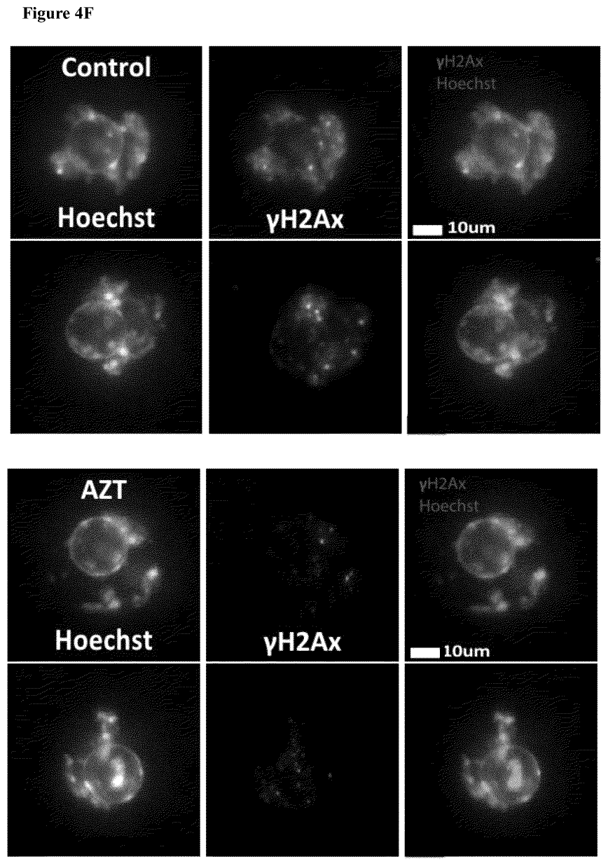

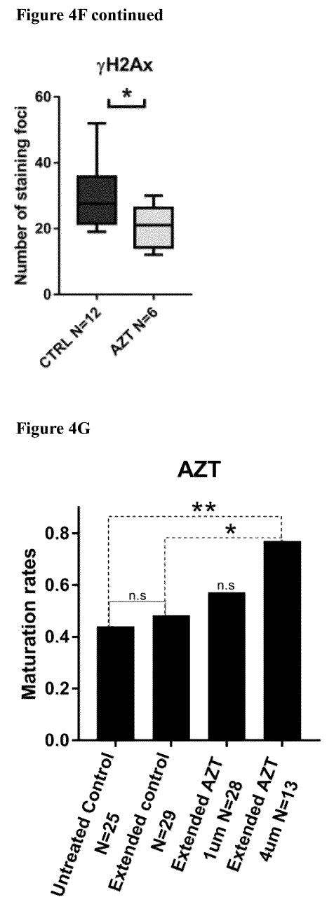

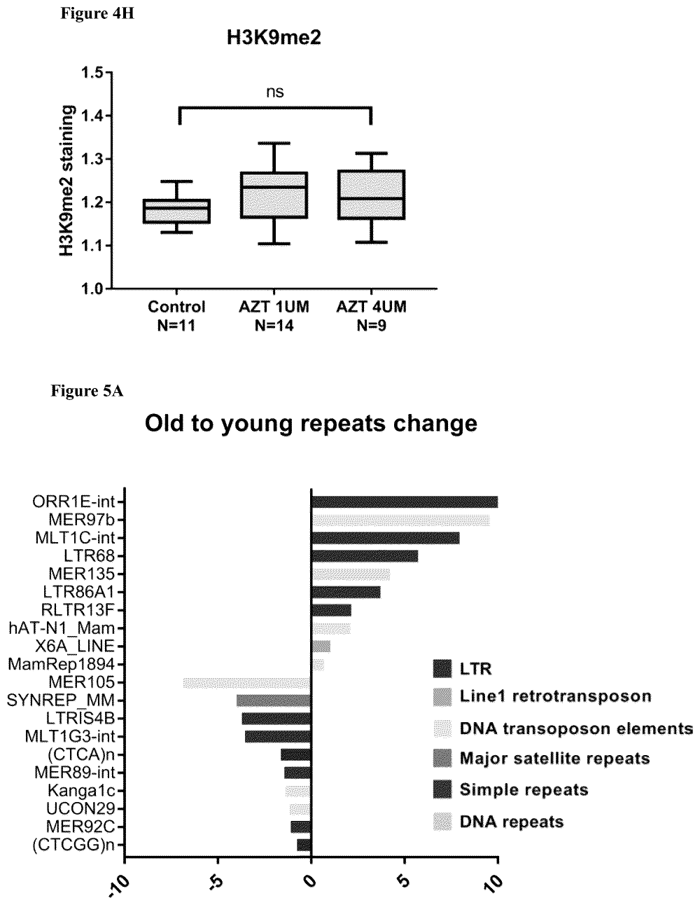

Histone post-translational modifications are a key component in transcriptional regulation, and specifically in the silencing of transposable elements (TE) that compose ˜50% of mammalian genomes. The activation of TE may pose a significant threat to the genome of the cell, and the regulation and silencing of the activity of TE is becoming recognized as a hallmark of cellular integrity in health and disease. To investigate the consequences of the loss of heterochromatin on the transcription of TE in old oocytes, we examined RNA expression from two retrotransposon families: long interspersed nuclear element-1 (L1) and intracisternal A particle (IAP), which were shown to be expressed in the germline. Oocytes from older females had a roughly 2-fold increased expression of both L1 and IAP transcripts compared with young oocytes as assessed by quantitative RT-PCR (FIG. 4A). An elevation in retrotransposon-derived protein synthesis in aged oocytes was detected by Western blot for a protein of the L1 retrotransposon, L1-encoded ORF1p (FIG. 4B). We confirmed these findings by immunostaining oocytes. L1-ORF1p was detectable at low levels in young oocytes and increased in intensity in older oocytes (FIG. 4C). In accordance with increased activity of these TE, older oocytes show increased recruitment of DNA repair machinery indicative of DNA damage. This was shown by elevated Rad51 nuclear localization in older oocytes and higher presence of γH2Ax foci (FIG. 4D-4E). Foci of γH2Ax are considered a bona fide marker of DNA damage and repair. An elevated nuclear localization of Rad51 is also a marker for DNA damage and was observed in HeLa and HCT116 cells after treatment with ionizing radiation.

We asked whether an inhibition of reverse transcription activity of retrotransposons, and as a result, inhibition of retrotransposition in older oocytes improves their cellular integrity and maturation ability. To do that, we matured in vitro older oocytes with or without the addition of azidothymidine (AZT), a reverse-transcriptase inhibitor, that was shown to inhibit retrotransposon activation in fetal oocytes. Being a thymidine kinase 1 inhibitor, AZT also presented a toxic effect on cell growth in some cell lines. However, previous studies on oocytes did not report a toxic effect in this system. Nevertheless, we used lower concentrations than those reported to be toxic (see Methods). Our results (FIG. 4G) show that the relatively low level of maturation of older oocytes in vitro can be partially rescued and show an elevation in maturation in up to 28.6% more mature oocytes after incubation in 4 μM AZT. We also show that DNA damage is also reduced by AZT treatment as presented by the reduction in γH2Ax loci in these oocytes (FIG. 4F). Despite these effects, the AZT treatment did not alter heterochromatin levels in the older oocytes (FIG. 4H), hinting that heterochromatin loss may be upstream to retrotransposons elevation.

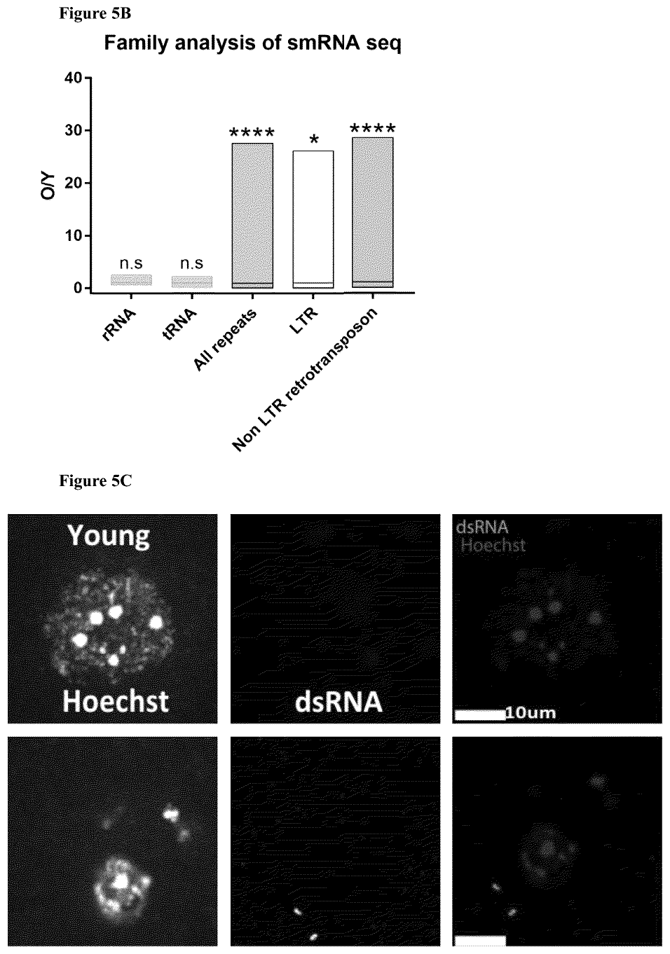

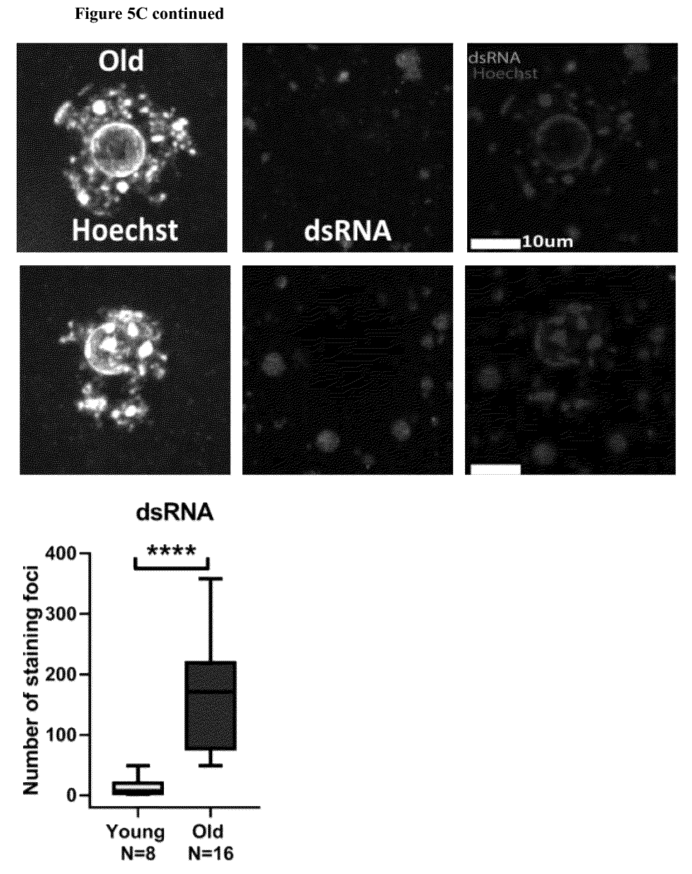

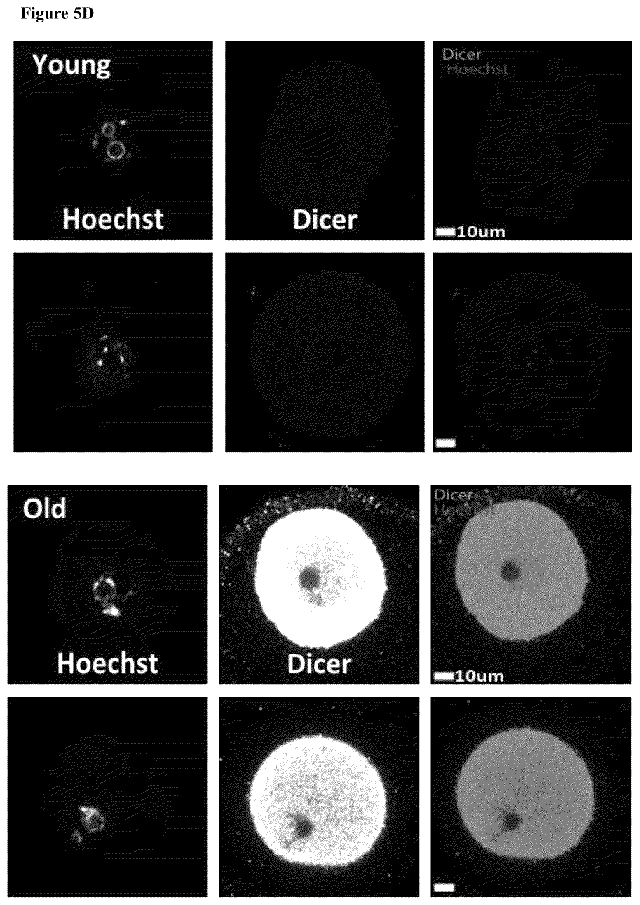

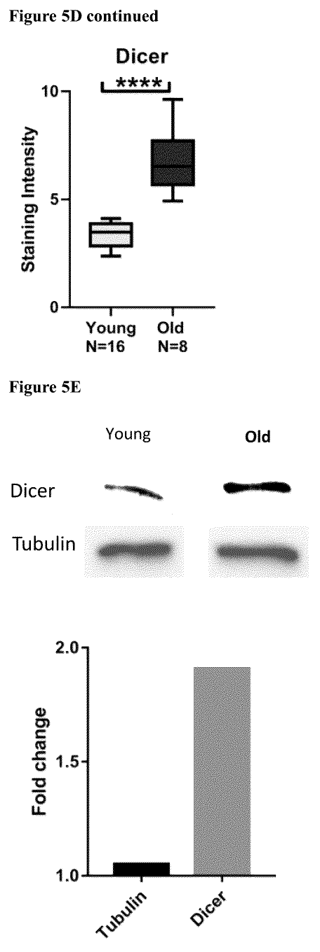

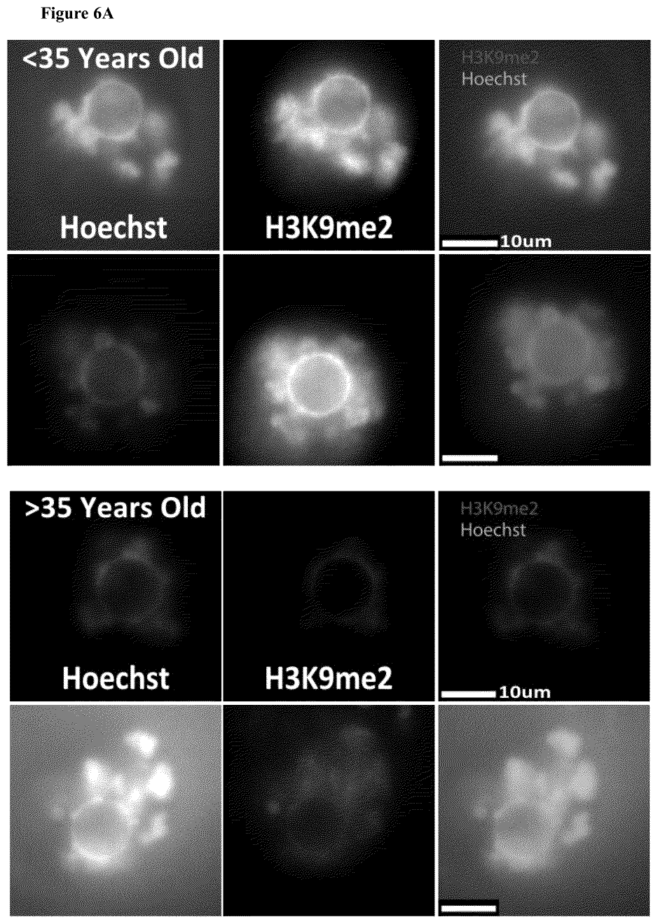

In addition to the expression of retrotransposons in older oocyte, we investigated whether we also see evidence for increased processing of retrotransposon RNA. An elevation in retrotransposon expression can result in elevated retrotransposon mRNA expression and its translation into protein but also in its elevated processing. RNA processing systems such as RNAi regulate gene expression by posttranscriptional targeting of mRNA coming from regulated regions. The processing system inhibits unregulated transcription activity by Dicer-mediated digestion of transcripts into small RNAs and activation of various downstream targeting and silencing complexes such as Argonaute proteins. Increased activity of the RNA processing machinery indicates the occurrence of a loss of transcription regulation, and it is possible to identify the specific loci where this loss has occurred by the analysis of the small RNA repertoire. We therefore performed small RNA sequencing on oocytes from young and old females. We sequenced (in duplicate) RNA molecules from 17 to 167 bp (median of 20-80 percentiles, see Methods) with a median molecule size of 18 bp for the young oocytes and 106 for the older oocytes. A measurement of the sizes of the small RNA fragments was done computationally (see Methods). A prominent peak of ribosomal RNA at 155 bp can be observed in both samples. The difference in small RNA sizes between young and old oocytes shows an elevated presence of RNA fragments of sizes that are not typical of Dicer products and could originate from spurious transcription due to the loss of genomic repression of heterochromatin. Small RNAs can originate from different regions in the genome. However, the difference in RNA expression between the young and old oocytes was evident specifically in the group of small RNAs coming from genomic repeats (usually marked by heterochromatin). Most repeat types remained unchanged between young and older oocytes (83%). 4.6% of repeat types were overexpressed and 12.1% were under-expressed more than 2-fold in older oocytes. However, when looking at which repeat types were changed, the list of overexpressed repeat types in old oocytes (FIG. 5A) shows that most RNA molecules are derived from retro-transposons, while in the under-expressed repeats additional repeat types are present. When we compared the fold change of expression in our data between different families of RNA molecules, we saw that while in tRNAs and rRNAs there is no elevation in older oocytes over young oocytes, in genomic repeats in general, and specifically in retrotransposons, there was a significant elevation in transcripts in older oocytes vs. young oocytes (FIG. 5B). In agreement with more retrotransposon mRNA and more retrotransposon-derived small RNA from some of the repeats in older oocytes, we also see a significant elevation in the signal of dsRNA molecules in older oocytes (FIG. 5C). Moreover, the Dicer protein, an enzyme which participates in the processing of retrotransposons showed an increase as well, both by immunostaining and Western blot analysis (FIG. 5D-5E). We therefore conclude that the loss of epigenetic silencing in old oocytes results in accompanied by transcriptional activity and processing of retrotransposons in the genome.

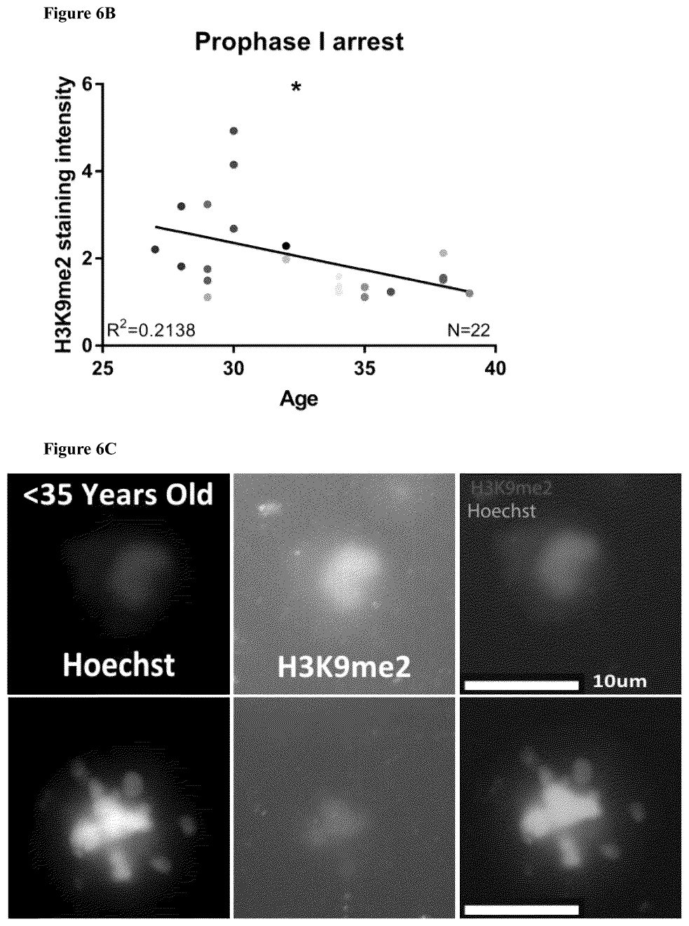

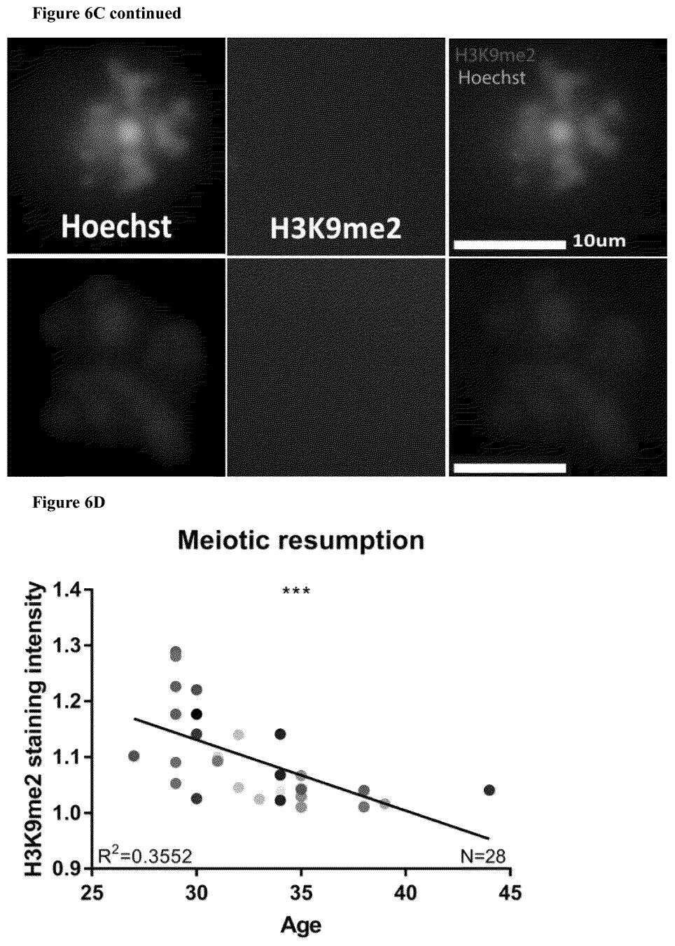

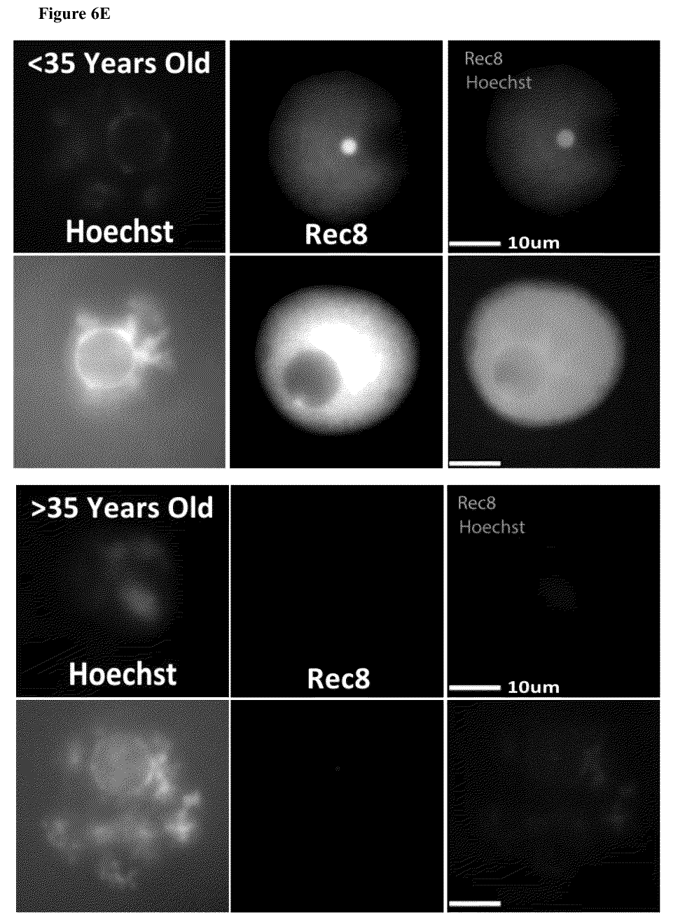

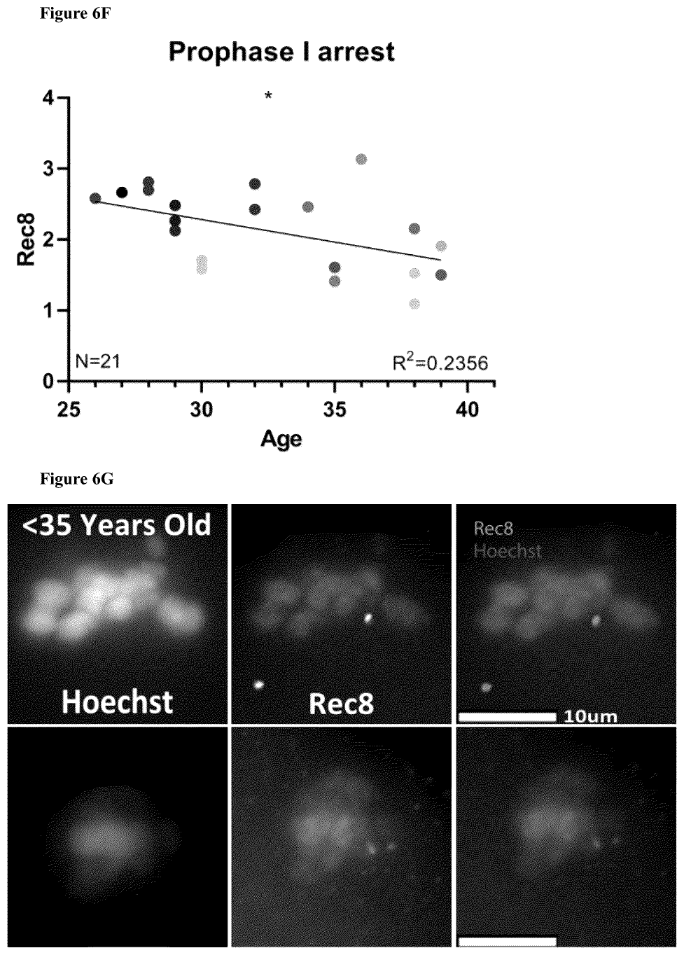

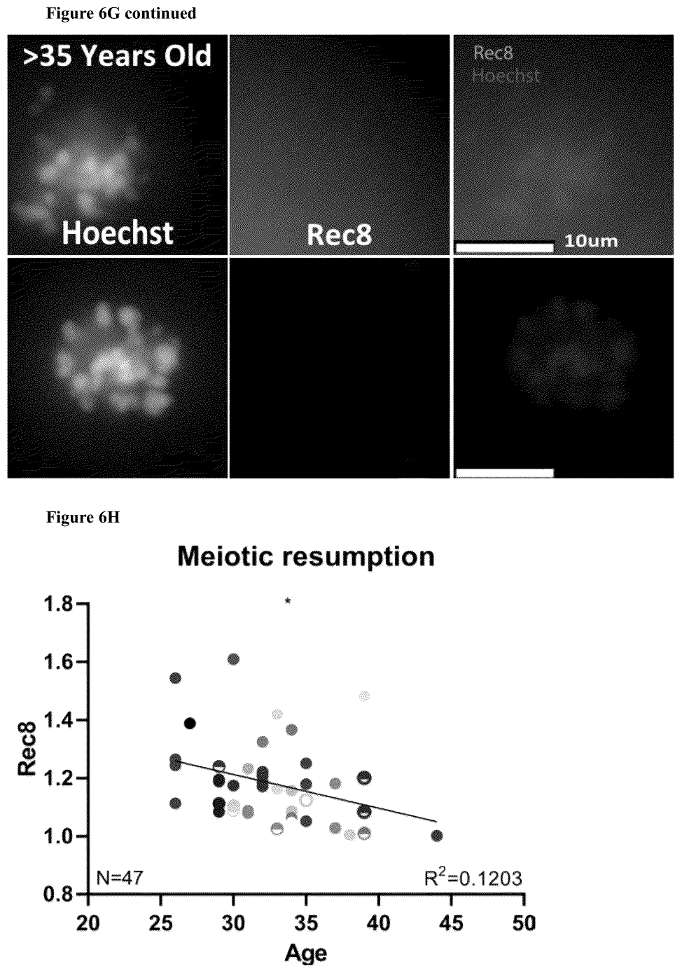

Since some significant differences in epigenetic regulation exist between mice and humans, we wanted to investigate whether heterochromatin loss with maternal age also occurs in human oocytes. For this purpose, we investigated the heterochromatin of oocytes from IVF treatments. Oocytes were retrieved from 33 patients, with an average of 2.2 oocytes per patient. As a general rule, prophase I-arrested oocytes are not used by clinics for fertilization, but to make sure the oocytes we used for research could not mature in vitro, we waited another 24 h with the oocytes in medium, before we fixed the immature oocytes and stained them. Therefore, these oocytes likely represent a subset of human oocytes which are meiotically incompetent. Indeed, fixed oocytes that were stained for DNA visualization were arrested in several developmental stages (FIG. 6). Further, we stained oocytes by in situ immunofluorescent staining for H3K9me2. Stringent QC criteria were applied to the staining results in order to exclude oocytes with fragmented or damaged genomes (see Methods). We found that in oocytes arrested both at the prophase I-arrest stage and after release from meiotic arrest, there was an age-dependent decrease in H3K9me2 signal fitting a linear decreasing curve (FIG. 6A-6D). As control, we also stained human oocytes for the Rec8 meiotic cohesin. This staining also shows (FIG. 6E-6H) a decreasing, age-dependent trend, as has been shown before for mouse and human cohesin.

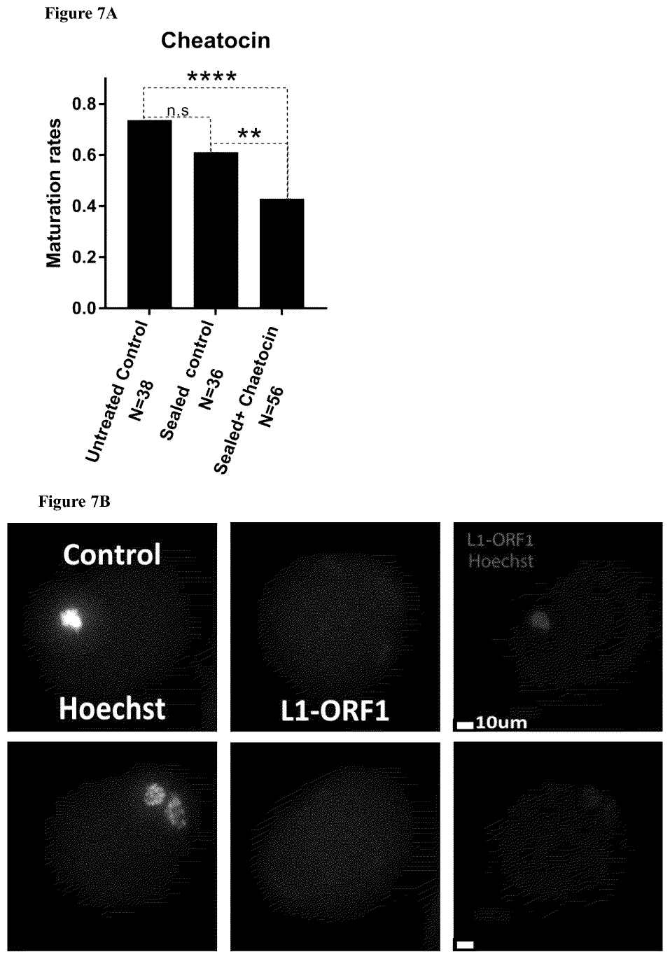

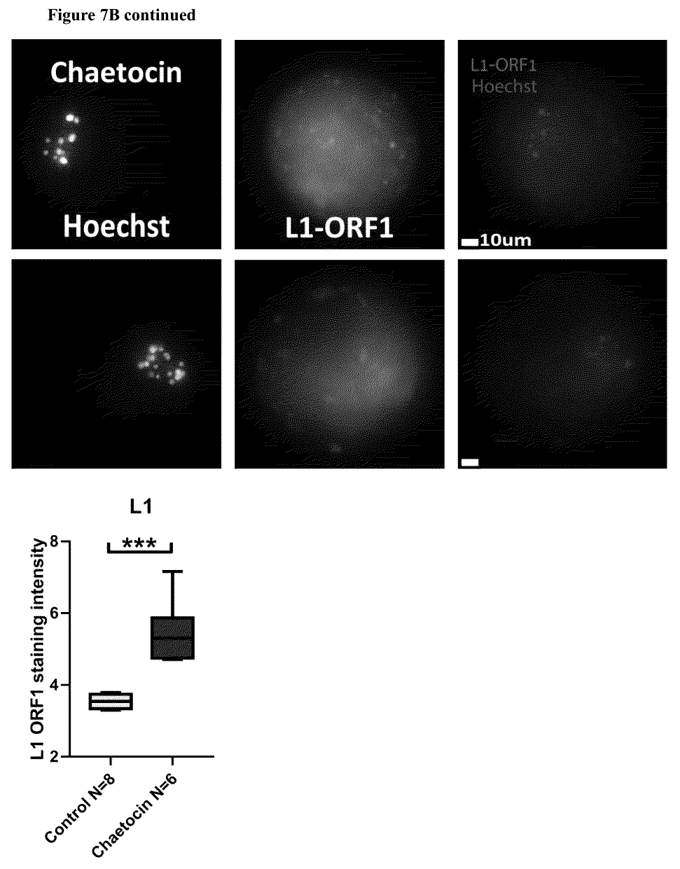

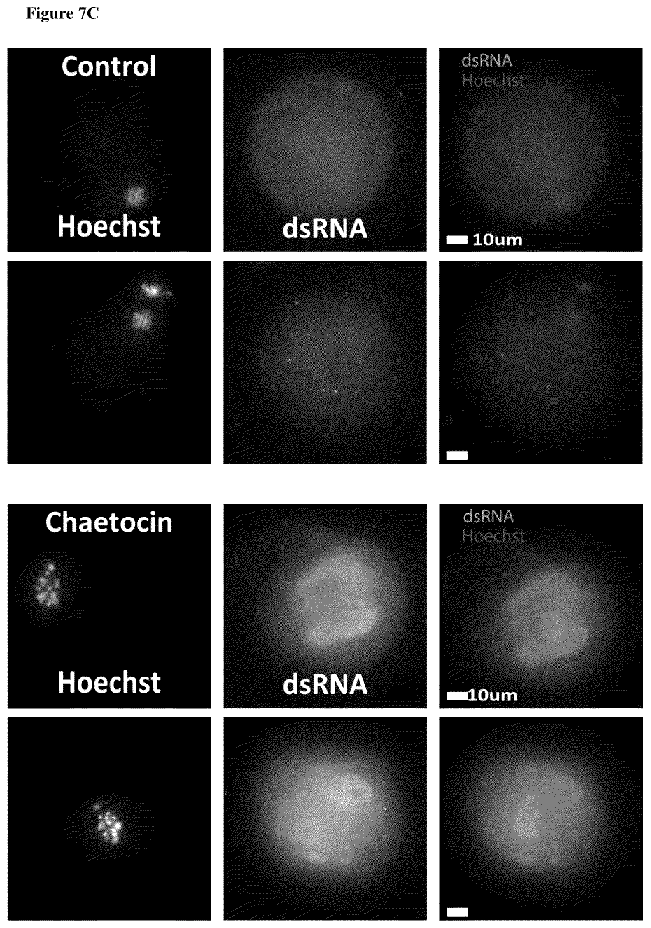

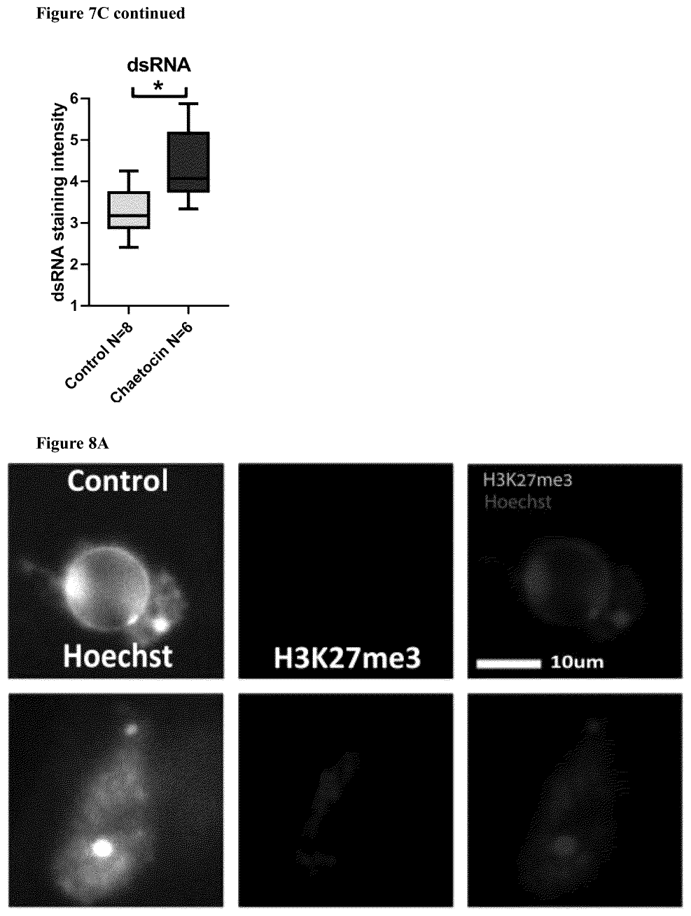

In order to show a causal link between heterochromatin deterioration and maturation defects of old oocytes, we treated young mouse oocytes with chemicals known to affect epigenetic regulation. The Suvar39h1/2 enzyme is essential for heterochromatin formation, and Chaetocin has been characterized as a specific and effective Suvar39h1/2 inhibitor. We therefore treated 2-month-old oocytes with Chaetocin at 0.5 μM in vitro for 18 h. Staining for H3K9me2 decreased after the treatment by staining of chromosome spreads at metaphase of MI. However, in situ staining of MI-treated oocytes for H3K4me3 presented no significant change, indicating that heterochromatin manipulation with Chaetocin does not interfere with other chromatin regulation aspects. The assessment of the maturation efficiency of the oocytes after treatment with Chaeotocin shows reduction of 18% in the oocytes that properly mature after treatment (FIG. 7A). Staining of treated oocytes by in situ immunofluorescence was performed after treatment. L1-encoded ORF1p shows an increase in staining intensity (FIG. 7B). Consistently, dsRNA staining presented a significant increase in the treated compared with the untreated control oocytes (FIG. 7C).

Since heterochromatin that is characterized by H3K9me2 and binding of HP1γ is also associated with a histone deacetylation on H3K27, we sought to investigate whether inhibiting the deacetylation of histones in oocytes will cause a similar effect. Previous reports also showed that treatment with a histone deacetylase (HDAC) inhibitor affects oocyte maturation in vitro. We thus treated young oocytes with the HDAC inhibitor Trichostatin A (TSA, 100 nm for 4 h of arrest and then for 18 h until MII). In vitro treated oocytes show an increase in H3K27Ac and also a decrease in H3K9me2 by in situ staining linking different heterochromatin pathways such as H3K27 deacetylation and H3K9 methylation in oocytes. TSA-treated young oocytes show similar maturation defects to Chaetocin-treated young oocytes and unchanged staining for H3K4me3. Interestingly, TSA-treated oocytes do not show a L1-encoded ORF1p elevation in overall signal level. Instead, L1-ORF1p accumulates in nuclei of treated oocytes, perhaps showing that deacetylation activity is central to specific stages in retrotransposon maturation processes, causing enhanced nuclear recruitment of the L1 protein when de-acetylation processes fail to occur. However, DNA damage response is elevated in TSA-treated oocytes as shown by the elevated Rad51 nuclear localization in treated oocytes. Collectively, these results show that epigenetic manipulation, in H3K9 methylation or H3K27 acetylation pathways causes the inability of young oocytes to mature, and mimics natural aging.

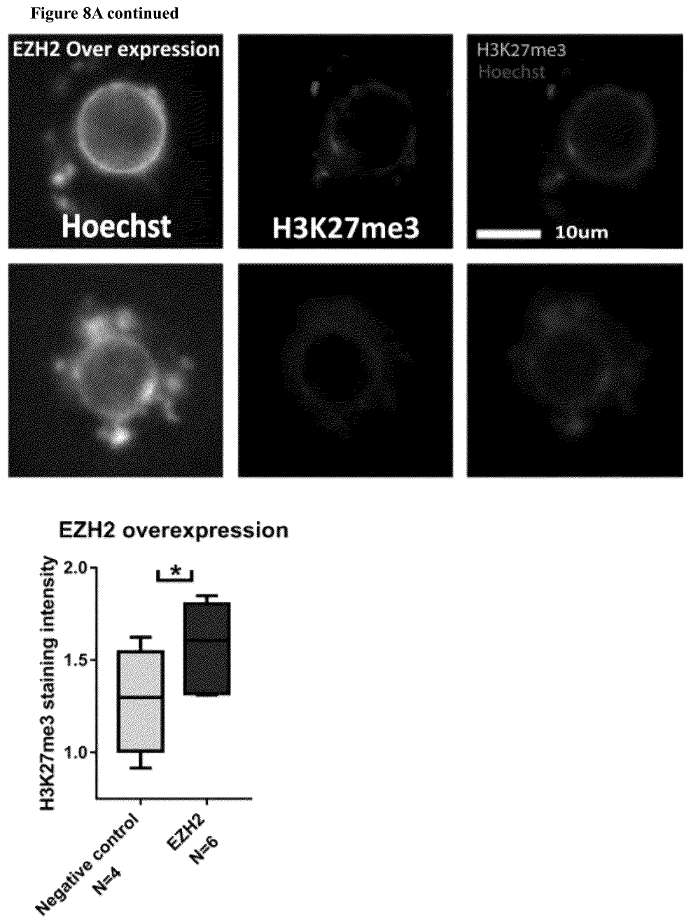

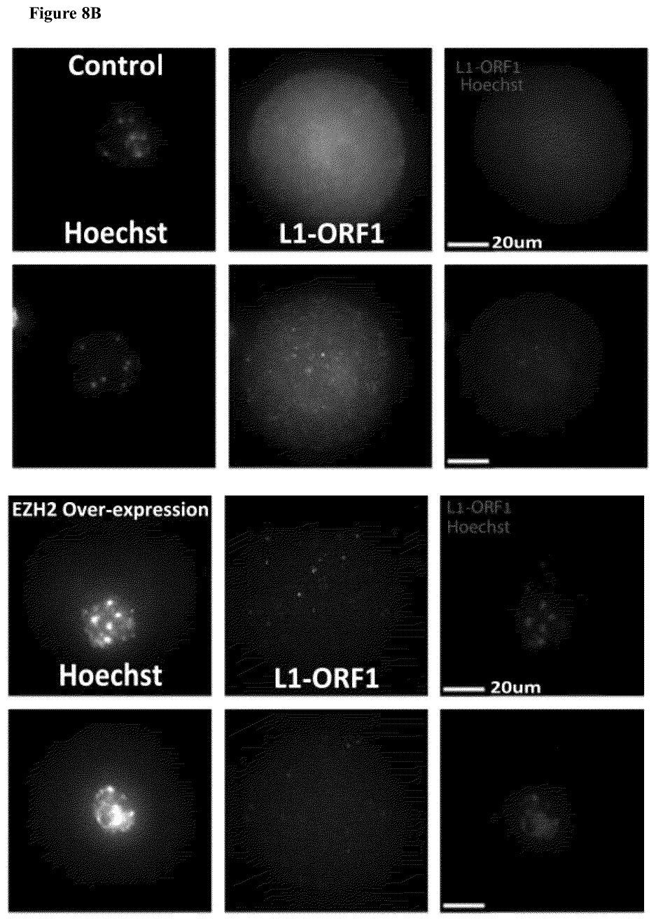

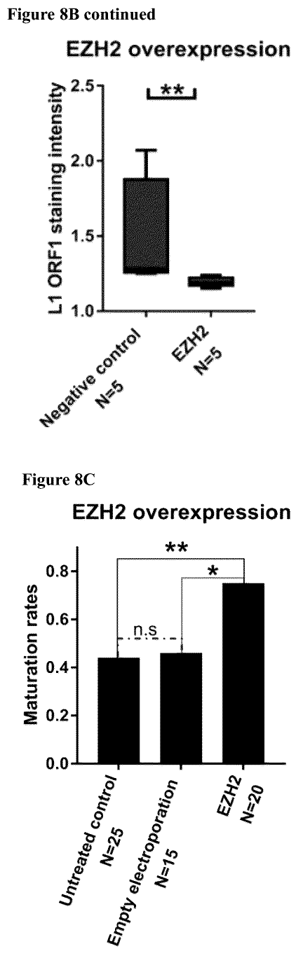

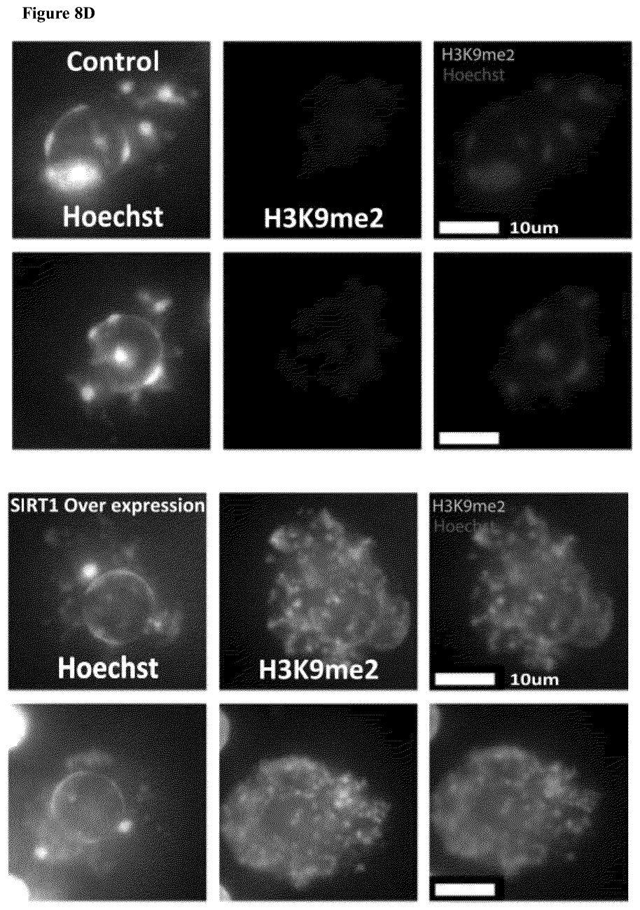

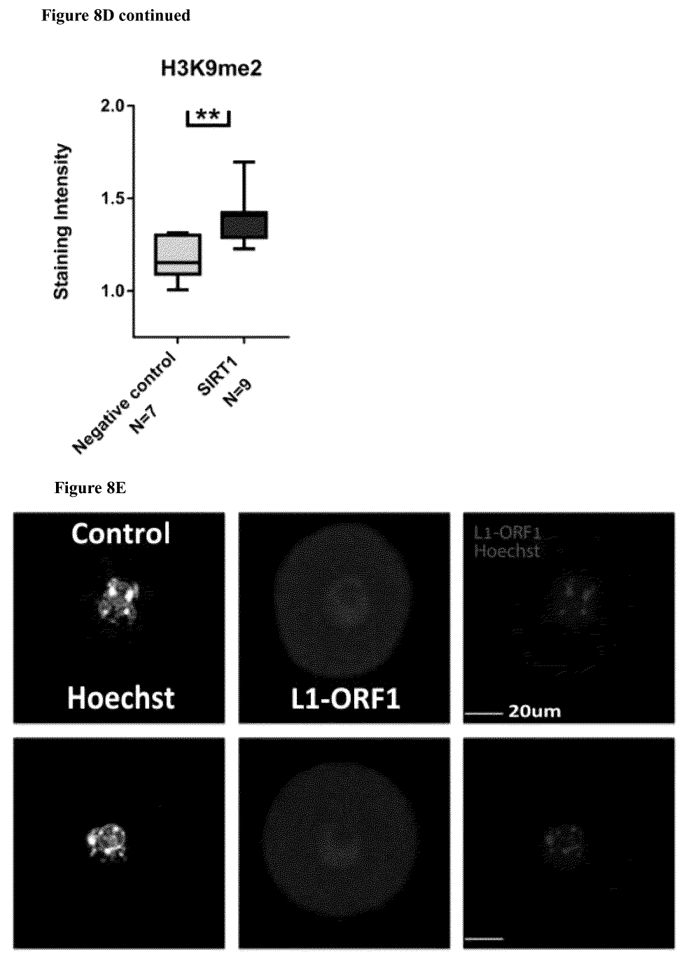

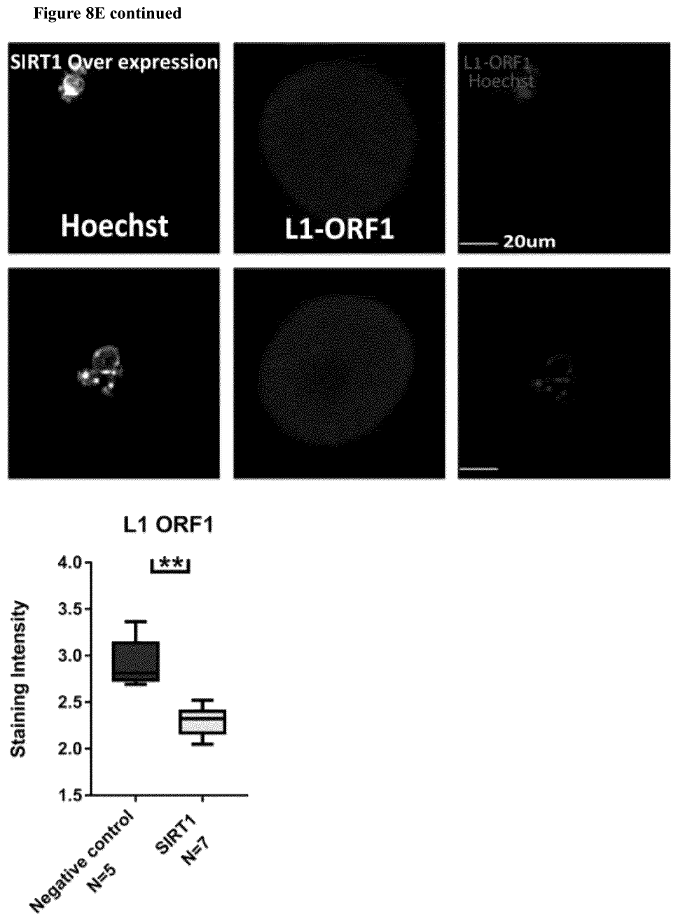

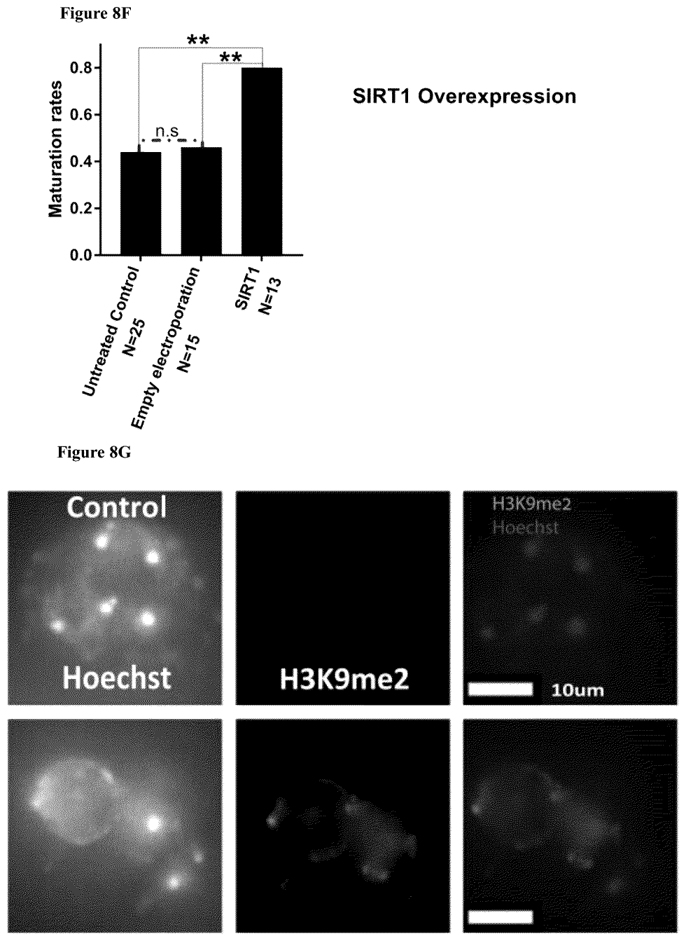

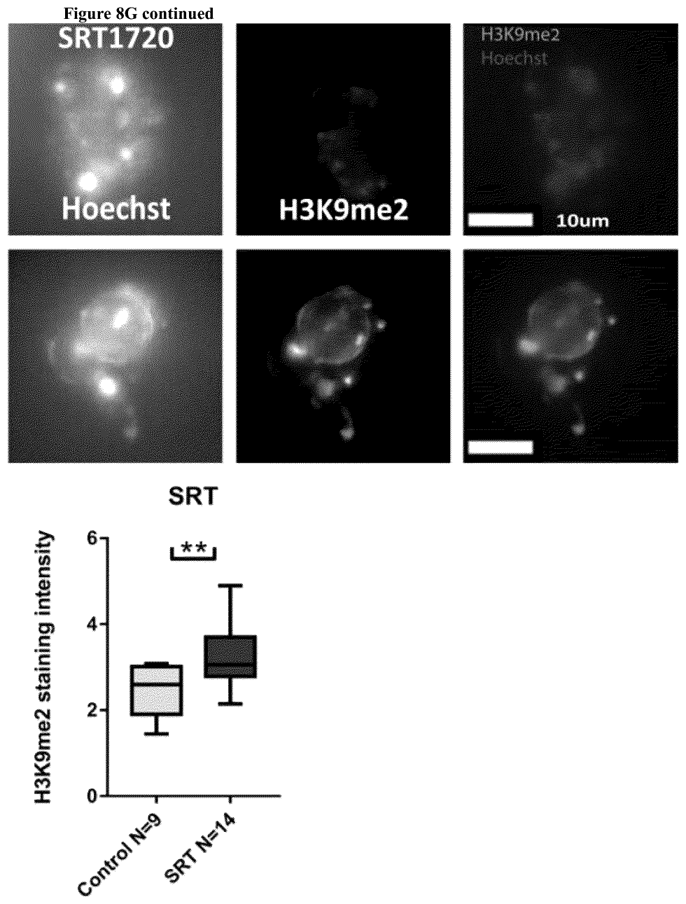

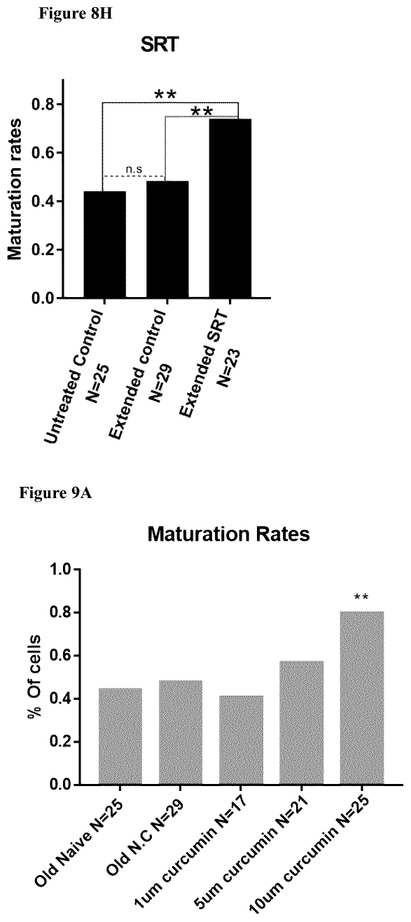

To further strengthen the causal link between heterochromatin loss, retroviral activity, and oocytes maturation defects in older oocytes, we tested whether the elevation of heterochromatin by gene overexpression could reverse oocyte aging phenotypes. To do that, we used plasmid electroporation as above, to overexpress EZH2, an enzymatic subunit of the PRC2 complex that catalyzes H3K27 methylation, and SIRT1, an NAD+dependent HDAC (see Methods). Treated oocytes presented a significant increase in heterochromatin. As expected, oocytes electroporated with EZH2 showed an elevation in H3K27 methylation staining compared with an empty electroporation control (FIG. 8A), and oocytes electroporated with SIRT1 plasmid presented an increase in H3K9 methylation. (FIG. 8D). In both groups, the increase in heterochromatin led to significant decrease in L1-ORF1 staining (FIG. 8B, 8E), showing that when heterochromatin is recovered in these oocytes they regain their ability to silence retroviral activity. Finally, treated oocytes presented increased maturation rates (FIG. 8C, 8F) compared with empty electroporation controls. To confirm these results with another manipulation targeting Sirtuins, we cultured aged oocytes with the SIRT1 activating molecule SRT-1720. Treated oocytes had a similar phenotype to the overexpression experiment above. SRT-1720 treatment triggered heterochromatin elevation, that resulted in recovered maturation ability of treated cells (FIG. 8G-8H). Notably, similar results were previously shown by supplementation of older oocytes with the SIRT1 associated metabolic cofactor nicotinamide adenine dinucleotide (NAD+), which caused rejuvenated oocyte quality in aged animals. Taken together, these results show that heterochromatin loss occurs upstream of retrotransposon activity, and that heterochromatin loss may be reversible in older oocytes.

### Example 2: IVM With HATIs and RTIs

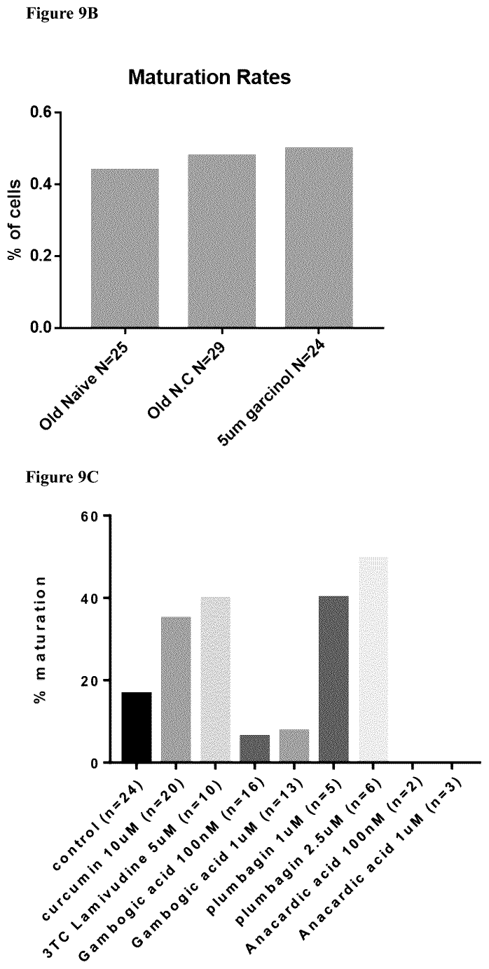

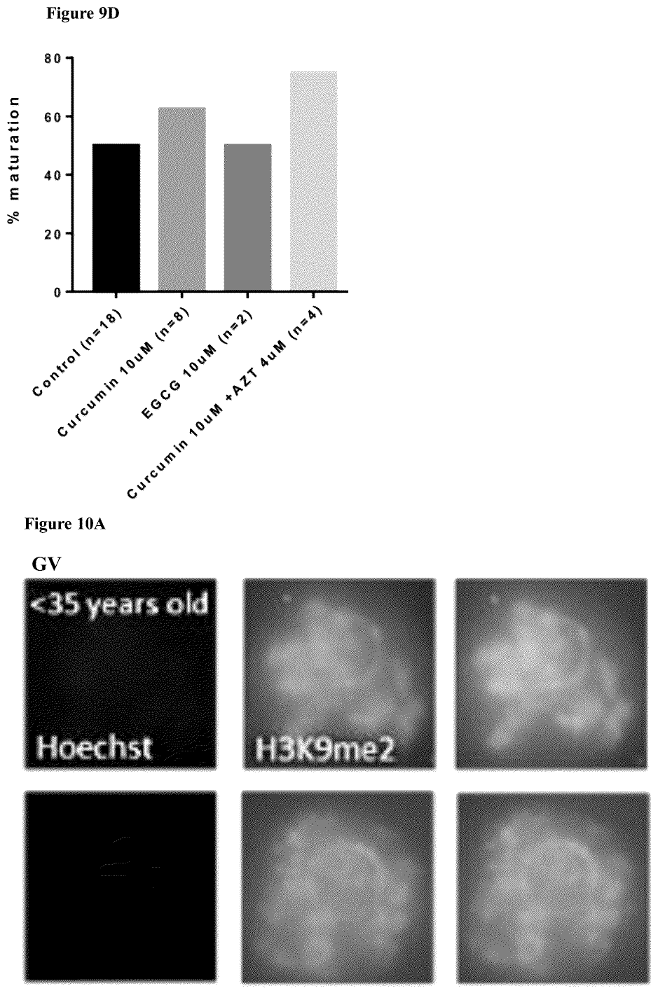

It was next tested if similar results could be achieved without genetic manipulation. Old oocytes were treated with two different naturally occurring histone acetyltransferase (HAT) inhibitors: curcumin and garcinol. As a negative control, oocytes were given vehicle only. Curcumin produces a dose dependent increase in maturation rate, such that at the highest dose (10 um) maturation rates over 80% were achieved (FIG. 9A). Garcinol also increased maturation rates, but to a lesser extent (FIG. 9B). Next, a broader panel of naturally occurring HAT inhibitors were tested along with another reverse transcriptase (RT) inhibitor (FIG. 9C). As before, old oocytes were incubated in IVM media (a-MEM) supplemented with the inhibitors for 24 hours and maturation was measured. Oocytes with both first polar body extrusion and proper division of chromosomes (as evaluated by Hoechst staining) were considered as mature. Curcumin was used as a positive control. The new RT inhibitor, Lamivudine (3TC) was found to be comparable to curcumin in its ability to induce maturation as was one of the HAT inhibitors, Plumbagin (FIG. 9C). Three other HAT inhibitors tested, gambogic acid and anacardic acid (FIG. 9C) and EGCG (FIG. 9D), did not produce increased maturation.

Gambogic acid, and anacardic acid are highly acidic and EGCG is more weakly acidic. There is evidence in the literature suggesting that lower pH levels are deleterious for oocyte maturation. The pH of the medium used was around 7.3 with buffering capacity between 6.9 and 7.6. The media did not have a particularly strong buffering capacity and so the addition of these three molecules rendered it acidic (more so for gambogic acid and anacardic acid). This acidity interfered with maturation and indeed in the case of the two strongly acidic molecules inhibited it. Medium with a greater buffering capacity or a different concentration of these acidic molecules will allow for HAT inhibition induced maturation improvement. It was further noted that gambogic acid and anacardic acid strongly inhibit PCAF while curcumin and plumbagin do not and that this may cause differences in maturation. Taken together, these results show that heterochromatin loss occurs upstream of retrotransposon activity, and that heterochromatin loss may be reversible in older oocytes, including by HAT inhibitors.

Finally, a combination of HAT inhibitor and RT inhibitor was tested. Old oocytes were cultured as before but in the presence of both curcumin and AZT and maturation was measured after 24 hours. Interestingly, the combination was superior to the effect of the HAT inhibitor alone (FIG. 9D). This is somewhat unexpected as heterochromatin loss is upstream of retrotransposon activity and so it might have been hypothesized that AZT would have no further effect once curcumin was added, however, a significant increase was observed with the combination.

### Example 3: Human Oocyte IVM With HATIs and RTIs

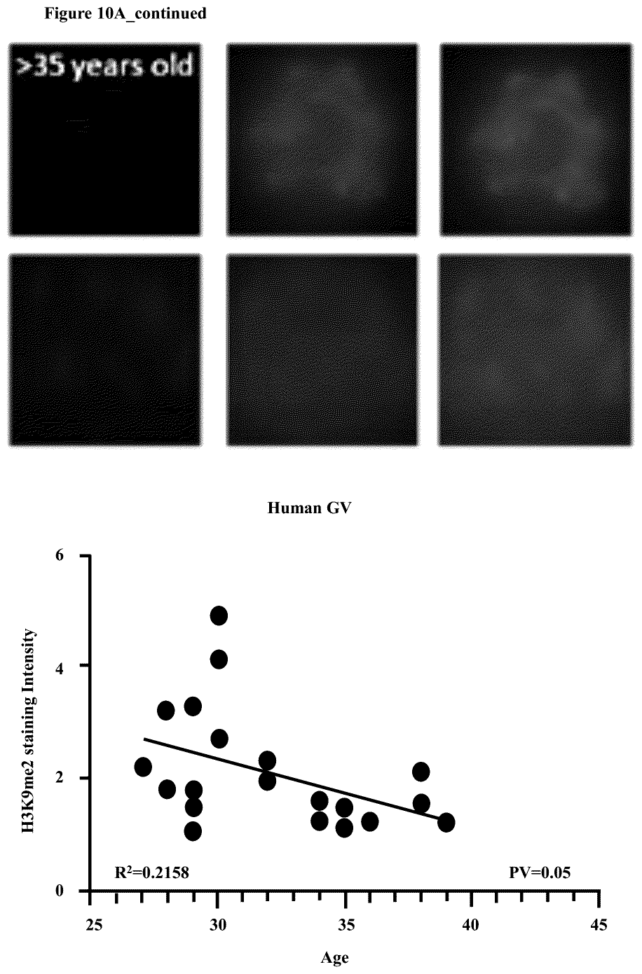

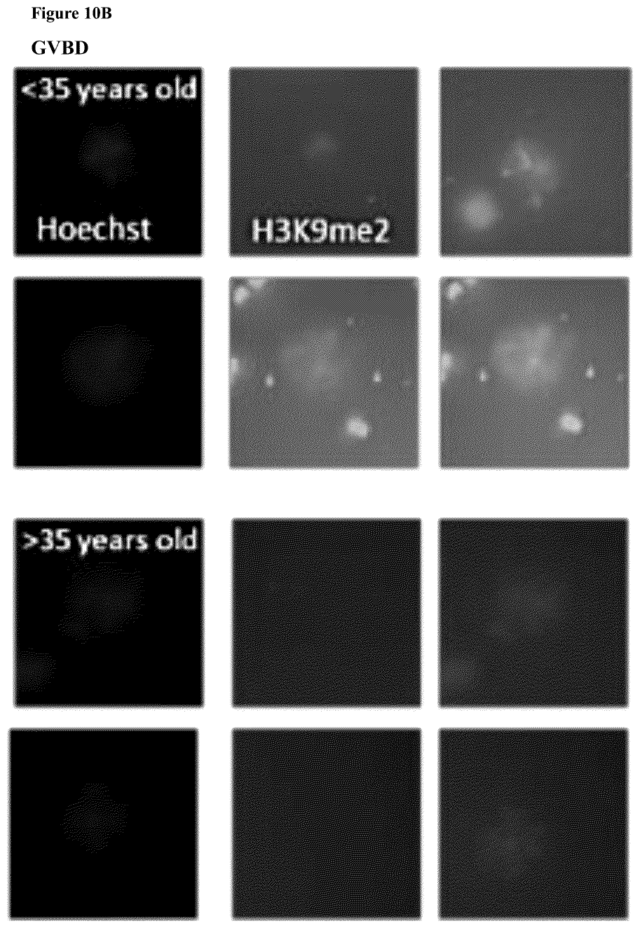

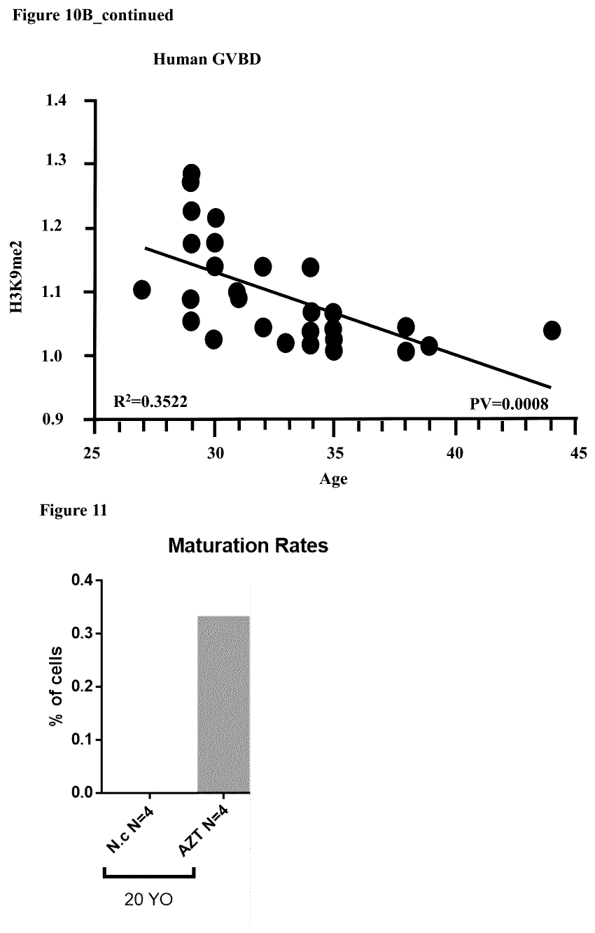

We wanted to investigate whether heterochromatin loss with maternal age is conserved in human oocytes. For this purpose, we investigated epigenetic markers of prematurely arrested oocytes from IVF treatments. Patients were admitted to the IVF clinic and stimulated by a protocol determined by their physician (see Methods). Antagonist protocol was started on the second day of the menstrual cycle. The specific dosage was determined individually for each patient according to BMI and ovarian reserve parameters. Gonadotropin releasing hormone (GnRH) antagonist or Human chorionic gonadotropin (hCG) or a combination of the two was administered for final oocyte maturation when at least 3 leading follicles were 17-18 mm in diameter. Oocyte retrieval was performed 36 hours following final maturation using transvaginal ultrasound guided approach. These oocytes (>150 um) represent a subset of human oocytes and when fixed and stained by Hoechst (DNA) they were indeed arrested in several developmental stages (FIG. 10A-10B).

Further, we stained oocytes by in-situ immunofluorescent staining for H3K9me2. Oocytes with fragmented or damaged genomes were excluded after analyzing the ratio between DNA and chromatin stain and using the shape of the genomic material. Our preliminary results show that in oocytes arrested both at the GV stage (with a GV clearly present) or after GV breakdown (GVBD), there was an age-dependent decrease in H3K9me2 signal fitting a linear decreasing curve (GV R2=0.21, GBVD R2=0.35, FIG. 10A-10B). As positive control, we also stained human oocytes for the Rec8 meiotic cohesin. This staining also shows a decreasing, age-dependent trend, as has been shown before for mouse and human cohesin. Thus, epigenetic deterioration and loss of heterochromatin also occur in human oocytes. Further, it should be noted that oocytes from some patients showed markedly less heterochromatin than would be expected for their age (dots in the lower left corner, below the trendline). Such woman have oocytes that are epigenetically older than the woman's biological age and would particularly benefit from the maturation methods provided herein.

To also look at non-arrested human oocytes, we investigated the behavior of another cohort of human oocytes extracted from patients undergoing gonadotoxic treatment and therefore undergoing fertility preservation. In these cases, when hormonal induction and oocyte extraction are not possible due to the young age of the patient or social reasons, ovary surgical extraction is performed. After extraction, ovaries are dissected in medium in the IVF lab, and ovary tissue is cryopreserved. After remission, ovary tissue can be transplanted, and IVF performed. During ovary dissection immature oocytes are released into the medium. These are usually discarded. After ethical committee approval and informed consent from patients, we retrieved oocytes from the medium at sizes of 150 μm and above and matured them in-vitro using Sage medium supplemented with 0.075 IU/mL luteinizing hormone (LH) and 0.075 IU/mL follicle stimulating hormone (FSH) overnight. 24-hours later denudation of oocytes was performed. Thereafter, assessment of polar body extrusion, chromatin status and immunostaining for epigenetic markers were performed. Incubation of oocytes from young ages with AZT, Garcinol, or Curcumin did not impair their maturation ability and even increased it (FIG. 11). Thus, immature oocytes can be matured in a superior manner by the supplementation of a HAT inhibitor, RT inhibitor or a combination thereof.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A cell culture medium comprising luteinizing hormone (LH), follicle-stimulating hormone (FSH) and at least one histone acetyltransferase (HAT) inhibitor or reverse transcriptase (RT) inhibitor, wherein said HAT inhibitor is selected from the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, anacardic acid, Gambogic acid, Delphinidin, Remodelin, butyrolactone 3 (MB-3), NK13650A, Taxodione (NP-15), Δ12-prostaglandin J2 (PGJ2), Plumbagin (RTK1), NU9056, DC-M01-7, CCT077791, NSC694614, PU141, C646, TH1834, L002, WM-8014, DC-G16-11, and derivatives thereof and said RT inhibitor is selected from the group consisting of: zidovudine, didanosine, zalcitabine, stavudine, lamivudine, abacavir, emtricitabine, entecavir, truvada, azvudine, tenofovir, adefovir, efavirenz, nevirapine, delavirdine, etravirine, rilpivirine, doravirine, islatravir and derivatives thereof.

2. The medium of claim 1, wherein said medium is human oocyte in vitro maturation (IVM) medium comprising at least HAT or RT inhibitor.

3. (canceled)

4. (canceled)

5. (canceled)

6. (canceled)

7. The medium of claim 1, wherein said HAT inhibitor is selected from curcumin, plumbagin and garcinol.

8. (canceled)

9. The medium of claim 1, wherein said RT inhibitor is selected from zidovudine (azidothymidine, AZT) and lamivudine (3TC).

10. The medium of claim 1, comprising both a HAT inhibitor and an RT inhibitor.

11. The medium of claim 10, comprising curcumin and AZT.

12. The medium of claim 1, wherein at least one of:
a. curcumin is present at a concentration of about 5-20 μM;
b. curcumin is present at a concentration of about 10 μM;
c. AZT is present at a concentration of about 0.5-5 μM;
d. AZT is present at a concentration of about 1 μM; and
e. said cell culture medium comprises a pH of between 6.8 and 7.8.

13. (canceled)

14. (canceled)

15. (canceled)

16. (canceled)

17. (canceled)

18. (canceled)

19. A method of in vitro maturing a human oocyte, the method comprising receiving an immature oocyte from a human subject, and culturing said received immature oocyte in culture medium comprising at least one HAT inhibitor or RT inhibitor for a time sufficient for first polar body extrusion from said oocyte, thereby in vitro maturing a human oocyte, where said HAT inhibitor is selected from the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, Epigallocatechin gallate (EGCG), Procyanidin-B3, anacardic acid, Gambogic acid, Delphinidin, Remodelin, butyrolactone 3 (MB-3), NK13650A, Taxodione (NP-15), Δ12-prostaglandin J2 (PGJ2), Plumbagin (RTK1), NU9056, DC-M01-7, CCT077791, NSC694614, PU141, C646, TH1834, L002, WM-8014, DC-G16-11, and derivatives thereof and said RT inhibitor is selected from the group consisting of: zidovudine, didanosine, zalcitabine, stavudine, lamivudine, abacavir, emtricitabine, entecavir, truvada, azvudine, tenofovir, adefovir, efavirenz, nevirapine, delavirdine, etravirine, rilpivirine, doravirine, islatravir and derivatives thereof.

20. The method of claim 19, wherein at least one of:
a. said human subject is at least 35 years or older;
b. said human subject is at least 38 years or older;
c. said human subject does not receive hormone injections before harvesting of said immature oocyte;
d. said culture media is IVM culture media supplemented with at least one gonadotropin; and
e. said culture media is IVM culture media supplemented with LH and FSH.

21. (canceled)

22. (canceled)

23. (canceled)

24. (canceled)

25. (canceled)

26. (canceled)

27. (canceled)

28. (canceled)

29. (canceled)

30. The method of claim 19, wherein said HAT inhibitor is selected from curcumin, plumbagin and garcinol.

31. (canceled)

32. The method of claim 19, wherein said RT inhibitor is selected from zidovudine (azidothymidine, AZT) and lamivudine.

33. The method of claim 19, wherein said culture media comprises both a HAT inhibitor and an RT inhibitor.

34. The method of claim 33, wherein said culture media comprises curcumin and AZT.

35. The method of claim 19, wherein at least one of:
a. said culture medium comprises a pH of between 6.8 and 7.8;
b. said culture medium is the culture medium comprises luteinizing hormone (LH), follicle-stimulating hormone (FSH) and at least one histone acetyltransferase (HAT) inhibitor or reverse transcriptase (RT) inhibitor, wherein said HAT inhibitor is selected from the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, anacardic acid, Gambogic acid, Delphinidin, Remodelin, butyrolactone 3 (MB-3), NK13650A, Taxodione (NP-15), Δ12-prostaglandin J2 (PGJ2), Plumbagin (RTK1), NU9056, DC-M01-7, CCT077791, NSC694614, PU141, C646, TH1834, L002, WM-8014, DC-G16-11, and derivatives thereof and said RT inhibitor is selected from the group consisting of: zidovudine, didanosine, zalcitabine, stavudine, lamivudine, abacavir, emtricitabine, entecavir, truvada, azvudine, tenofovir, adefovir, efavirenz, nevirapine, delavirdine, etravirine, rilpivirine, doravirine, islatravir and derivatives thereof;
c. said time is about 18-72 hours;
d. said HAT inhibitor is present at a concentration sufficient to increase constitutive heterochromatin levels in said oocyte by at least a predetermined threshold;
e. said HAT inhibitor or RT inhibitor is present at a concentration sufficient to decrease retrotransposon expression in said oocyte by at least a predetermined threshold;
f. said method elevates maturation rates as compared to IVM performed in the absence of said HAT inhibitor or said RT inhibitor; and
g. said HAT inhibitor or RT inhibitor is present at a concentration sufficient to increase first polar body extrusion as compared to culturing in media devoid of said HAT inhibitor or RT inhibitor.

36. (canceled)

37. (canceled)

38. (canceled)

39. (canceled)

40. (canceled)

41. (canceled)

42. (canceled)

43. (canceled)

44. The method of claim 19, wherein said culturing further comprises the addition of a meiosis entry inhibitor.

45. (canceled)

46. (canceled)

47. A method of determining suitability of a subject in need of IVM of said subject's oocyte to undergo the method of claim 19, the method comprising:
a. receiving from a human female subject at least one germinal vesicle (GV)-arrested oocyte; and
b. measuring heterochromatin in said GV-arrested oocyte, wherein a heterochromatin level below a predetermined threshold indicates the subject is suitable for IVM of said subject's oocyte;
thereby determining the suitability of a subject's oocyte.

48. The method of claim 47, wherein said GV-arrested oocyte was harvested for in vitro fertilization (IVF) but was found insufficiently mature.

49. The method of claim 47, wherein said measuring heterochromatin comprises measuring histone 3 lysine 9 (H3K9) trimethylation, H4K20 trimethylation, heterochromatin protein 1 (HP1) levels, 5 methylcytosine levels or a combination thereof; said subject has undergone at least one unsuccessful round of IVF and heterochromatin levels above said predetermined threshold indicate said subject is suitable for another round of IVF or both.

50. (canceled)

51. A kit comprising:
a. cell culture medium;
b. luteinizing hormone (LH) and follicle-stimulating hormone (FSH); and
c. at least one histone acetyltransferase (HAT) inhibitor or reverse transcriptase (RT) inhibitor, wherein said HAT inhibitor is selected from the group consisting of: Curcumin, Garcinol, Spermidinyl-coenzyme A (Spd-CoA), Lys-CoA, Histone H4 lysine 16 (H4K16)-CoA, MG149, EML264, LTK-14, Procyanidin-B3, anacardic acid, Gambogic acid, Delphinidin, Remodelin, butyrolactone 3 (MB-3), NK 13650A, Taxodione (NP-15), Δ12-prostaglandin J2 (PGJ2), Plumbagin (RTK1), NU9056, DC-M01-7, CCT077791, NSC694614, PU141, C646, TH1834, L002, WM-8014, DC-G16-11, and derivatives thereof and said RT inhibitor is selected from the group consisting of: zidovudine, didanosine, zalcitabine, stavudine, lamivudine, abacavir, emtricitabine, entecavir, truvada, azvudine, tenofovir, adefovir, efavirenz, nevirapine, delavirdine, etravirine, rilpivirine, doravirine, islatravir and derivatives thereof.

52. The kit of claim 51, wherein at least one of:
a. said medium is human oocyte in vitro maturation (IVM) medium;
b. said HAT inhibitor is selected from curcumin, plumbagin and garcinol;
c. said RT inhibitor is selected from zidovudine (azidothymidine, AZT) and lamivudine (3TC);
d. said kit comprises both a HAT inhibitor and an RT inhibitor; and
e. said kit comprises curcumin and AZT.

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)
